(19) United States
(12) Patent Application Publication
(10) Pub. No.: US 2016/0271321 A1
(43) Pub. Date: Sep. 22, 2016

(54) DIRECTIONAL SHEATH AND RELATED METHOD OF USE

(71) Applicant: Spectrum Health Innovations, LLC, Grand Rapids, MI (US)

(72) Inventors: Christopher Michael Chambers, Grand Rapids, MI (US); Louis Johan-Anton Weijers, Belmont, MI (US)

(21) Appl. No.: 15/071,694

(22) Filed: Mar. 16, 2016

Related U.S. Application Data
(60) Provisional application No. 62/134,307, filed on Mar. 17, 2015.

(51) Int. Cl. A61M 5/00; A61B 6/00; A61M 25/10

(52) U.S. Cl. CPC A61M 5/007; A61M 25/1006; A61M 25/1018; A61B 6/481; A61B 6/485; A61B 6/504; A61M 2025/105

(57) ABSTRACT

A directional sheath for use in angiography, thrombectomy and/or chemotherapy procedures. The sheath includes a distal end and proximal end, defines aperture(s) between the ends, and includes an occlusion balloon adjacent the distal end. An inflation lumen is in fluid communication with the balloon, so the balloon can be inflated to occlude a blood vessel. The sheath can include a dilator. In one method, the sheath is inserted in a blood vessel, and the balloon is inflated to occlude it. A substance is introduced through the sheath, while the distal end remains occluded by a dilator distal end. The substance passes a dilator intermediate portion and exits the sheath through the apertures, whereby the substance is introduced retrograde in the blood vessel. In another method, the sheath is introduced into a vessel having a thrombus, the balloon is inflated to occlude it, and the thrombus is pulled into the sheath.

### BACKGROUND OF THE INVENTION

The present invention relates to introducer sheaths for use in medical procedures involving blood vessels, and more particularly to introducer sheaths for use in connection with angiography, thrombectomy and other similar procedures.

Angiography, also referred to as arteriography, is a medical imaging technique used by health care providers to visualize blood vessels and organs of the body, for example, arteries, veins and heart chambers. In the process, a radio-opaque contrast agent, also referred to as a contrast dye, is injected into the blood vessel. The blood vessel is then imaged using X-ray based techniques, such as fluoroscopy. In the resulting image, the contrast dye is readily visible, and can be used to visualize the structure and integrity of the blood vessel.

Angiography can be used to evaluate a variety of conditions. For example, subjects undergoing dialysis may need to have a particular type of angiogram, an arteriovenous (AV) fistulogram, to enable a health care provider to search and possibly identify damage to a fistula, which is a connection between a vein and an artery of the subject. This type of angiogram can also enable the health care provider to identify and evaluate stenosis, which is a narrowing of the vein, adjacent the fistula or elsewhere.

During angiography, a health care provider typically uses a radial sheath introducer to access the blood vessel and introduce the contrast dye. A radial sheath introducer includes an introducer sheath and a dilator disposed inside the sheath. The dilator and sheath are inserted into a subject's blood vessel. The dilator is removed, and contrast is then injected though the sheath into the subject's blood vessel, typically in the direction of blood flow.

In some angiography procedures, it is desirable to inject the contrast retrograde, that is, counter to the flow of blood within the blood vessel. This is common in fistulograms. In many cases, a health care provider wants to see if there is damage to the blood vessel, typically a vein, upstream of the location where the sheath is introduced to the vein. In addition, the health care provider may want to verify the condition of the fistula. To view upstream, either a new introducer sheath is inserted upstream, or the vein is manually occluded by way of the health care provider applying external pressure on the subject's arm, which in turn physically compresses and pinches off the vein. The dye from the introducer is then introduced retrograde into the vein and imaged.

An issue with current retrograde angiograms is that it takes extensive skill for the health care provider to perfectly occlude the vein upon application of external forces to the subject's arm. Sometimes, the vein will move under the skin, unbeknownst to the provider. Thus, when the contrast dye is introduced, it will not adequately enter the upstream area of the vein desired to be imaged, and the opportunity to view this area is compromised. In some cases, the difficulty of viewing retrograde can significantly increase procedure time, from twenty minutes to as much as one hour. This can needlessly consume time in the lab that could be spent on other procedures.

Retrograde angiography procedures also typically require more nurses to be in the room for the procedure, which can significantly increase cost. In addition, throughout the procedure, the subject is being viewed with fluoroscopy, so any increase in the procedure duration results in additional exposure to radiation. Further, the provider's hand is usually exposed to radiation for the duration of the fistulogram due to the provider's manual occlusion of the vein. Lastly, manual occlusion of the vein and significantly larger injection pressures may unintentionally rupture the subject's vein. This can increase procedure time and also increase the recovery time for the subject.

### SUMMARY OF THE INVENTION

A directional sheath for use in angiography, thrombectomy and similar procedures is provided. The sheath includes a distal end and proximal end, defines one or more apertures through which fluid exits the sheath, located between the ends, and includes an occlusion balloon adjacent the distal end.

In one embodiment, the sheath includes an inflation lumen that is in fluid communication with the occlusion balloon, so that the balloon can be inflated to occlude a blood vessel. The inflation lumen can extend from the occlusion balloon to the proximal end of the sheath. There, the sheath can include an inflation port adapted to selectively introduce fluid into the inflation lumen to subsequently inflate the occlusion balloon. When inflated in a blood vessel, the occlusion balloon selectively occludes that blood vessel.

In another embodiment, the sheath includes a dilator. The dilator can include a proximal end, and a distal end which is located at least partially in the sheath. The distal end can have a first dimension, such as a diameter, selected so that the distal end can occlude a sheath distal end opening, thereby preventing fluid in the sheath from exiting there.

In still another embodiment, the dilator can include an intermediate portion between the distal end and the proximal end, the dilator intermediate portion can have a second dimension, such as a diameter, selected so that fluid within the sheath can flow in a fluid passageway defined between the dilator and an interior surface of the sheath, subsequently out at least one of the apertures, distal from the distal end of the sheath. The second dimension can be less than the first dimension.

In yet another embodiment, the dilator intermediate portion can include a dilator passageway, extending along a longitudinal axis of the dilator. The dilator passageway can be in the form of a recess extending helically about the dilator longitudinal axis. The passageway can enable fluid within the sheath to flow between the dilator, in the recess, and an interior surface of the sheath, subsequently out at least one of the apertures, distal from the distal end of the sheath.

In even another embodiment, the sheath can define an internal sheath lumen bounded by an exterior sheath wall. The dilator can be selectively disposed in this internal sheath lumen.

In still another embodiment, the exterior sheath wall can define an injection lumen separate and offset from the internal sheath lumen. The injection lumen can be in fluid communication with the one or more apertures defined by the sheath, so that fluid, such as contrast dye, can be conveyed through the injection lumen and out the apertures, without ever entering the internal sheath lumen.

In yet another embodiment, the dilator can be rotatably positioned in the internal sheath lumen. The dilator can include a relief surface, adjacent which a fluid passageway is defined. The dilator can be selectively rotated to align the passageway with the one or more apertures, so that liquid can flow through the passageway and out the apertures, into a blood vessel.

In a further embodiment, the sheath includes a dilator having a proximal end, and a distal end at least partially located in the sheath. Another portion of the distal end can extend beyond the sheath, out a sheath end opening. That other portion can include a dilator occlusion balloon. When inflated, the dilator occlusion balloon can occlude a blood vessel within which the sheath is placed.

In still a further embodiment, the dilator can define a dilator internal lumen. The dilator internal lumen can be separate and distinct from a dilator guide wire lumen. The dilator internal lumen can be in fluid communication with and can extend to a dilator proximal end, at which it can be placed in fluid communication with a fluid supply sufficient to inflate the occlusion balloon.

In a yet a further embodiment, an angiography procedure is provided. The method can include one or more of the following steps: providing an introducer sheath defining a plurality of apertures between a sheath distal end and a sheath proximal end, the introducer sheath including an occlusion balloon located adjacent the sheath distal end; providing a dilator including a dilator distal end and a dilator proximal end, with a dilator intermediate portion located therebetween, the dilator positioned so that the dilator distal end occludes the sheath distal end and so that liquid cannot flow out the sheath distal end; inserting the sheath distal end into a blood vessel of a subject; inflating the occlusion balloon so that the balloon occludes the blood vessel; and introducing an angiography dye into the introducer sheath so that the angiography dye exits the introducer sheath through the plurality of apertures whereby the angiography dye is introduced retrograde in the blood vessel.

In even a further embodiment, a thrombectomy procedure is provided. The method can include one or more of the following steps: providing an introducer sheath including a sheath distal end and a sheath proximal end, the introducer sheath defining a plurality of apertures between the sheath distal end and the sheath proximal end, the introducer sheath including an occlusion balloon located adjacent the sheath distal end; providing a dilator including a dilator distal end and a dilator proximal end, the dilator being selectively disposed in the introducer sheath; inserting the sheath distal end into a blood vessel of a subject, the blood vessel having a thrombus therein; inflating the occlusion balloon so that the balloon occludes the blood vessel; removing the dilator from the introducer sheath; and applying a vacuum via the introducer sheath so that at least a portion of the thrombus enters the introducer sheath and is removed from the blood vessel.

In another further embodiment, another procedure is provided. The method can include one or more of the following steps: providing an introducer sheath including a sheath distal end and a sheath proximal end, the introducer sheath defining a plurality of apertures between the sheath distal end and the sheath proximal end, the introducer sheath including an occlusion balloon located adjacent the sheath distal end; inserting the sheath distal end into a blood vessel of a subject; inflating the occlusion balloon so that the balloon occludes the blood vessel; and conveying fluid through the sheath, optionally between a dilator and an interior sheath surface of the sheath.

The current embodiments of the directional sheath and related methods herein provide a variety of advantages. For example, when used in conjunction with a retrograde angiography procedure, the directional sheath and its method of use can significantly reduce angiography procedure time to free up the lab to perform other procedures. Additionally, with the improved ease of use, fewer nurses can be present in the room for the procedure. With the directional sheath used in conjunction with the angiography, a healthcare provide no longer needs to manually occlude a blood vessel for the duration of the procedure. This in turn reduces the provider's exposure to radiation during the angiography. Further, without manual occlusion of the blood vessel, injection pressures can be reduced, and thus the possibility of rupturing a subject's blood vessel can be reduced.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a perspective view of a current embodiment of a directional sheath being used in an angiography procedure upon initial installation of the directional sheath in a blood vessel;

FIG. 2 is a close up partial section view of the directional sheath in the blood vessel;

FIG. 3 is a perspective view of the directional sheath including an occlusion balloon being inflated to occlude the blood vessel;

FIG. 4 is a close up partial cross section view of the occlusion balloon occluding the blood vessel and altering blood flow;

FIG. 4A is a close up cross section of a distal end of the directional sheath with an associated occlusion inflated;

FIG. 5 is a perspective view of a contrast dye syringe being attached to a port, with the port in the off position;

FIG. 6 is a perspective view of the contrast dye syringe injecting contrast dye through an open port in the open position, into the directional sheath and out the aperture as defined by the directional sheath;

FIG. 7 is a close up partial cross section of the directional sheath being used to introduce the contrast dye retrograde into the blood vessel;

FIG. 8 is a perspective view of the contrast dye continuing to be introduced retrograde into the blood vessel;

FIG. 9 is a cross section view of the directional sheath in the blood vessel taken along lines IX-IX of FIG. 7;

FIG. 10 is a cross section view of the blood vessel and sheath being taken along lines X-X of FIG. 7;

FIG. 11 is a perspective view of a dilator being removed from the directional sheath, with the contrast dye syringe and port in an off position;

FIG. 12 is a perspective view of the contract dye port in an on position, with the contrast dye syringe injecting contrast dye in the direction of natural blood flow into the blood vessel;

FIG. 13 is a cross section of a first alternative embodiment of the directional sheath illustrating an internal sheath lumen, and a separate injection lumen and inflation lumen;

FIG. 14 is a side view of a second alternative embodiment of the directional sheath illustrating a dilator having a helical or spiral channel adapted for conveying fluid to holes defined by the outer wall of the sheath;

FIG. 15 is a side view of a third alternative embodiment of the directional sheath including a rotational dilator having a removed portion;

FIG. 16 is a section view of the directional sheath taken along lines XVI-XVI of FIG. 15, wherein the dilator prevents fluid from exiting an aperture defined by the outer wall of the sheath;

FIG. 17 is a side view of the directional sheath of a third alternative embodiment with the dilator rotated to expose a relief surface and establish a fluid passageway to the holes defined the sheath outer wall;

FIG. 18 is a cross section view taken along lines XVI-XVI of FIG. 15, however, the dilator has been rotated so that a fluid passageway or fluid channel is established to enable fluid to flow out the hole that is defined by the sheath outer wall;

FIG. 19 is a perspective view of an adjuster adapted to rotate the dilator;

FIG. 20 is a partial section view of a fourth alternative embodiment of the directional sheath having an occlusion balloon directly joined with a dilator;

FIG. 21 is a close up, partial section view of a directional sheath of a fifth alternative embodiment, within a blood vessel during a thrombectomy;

FIG. 22 is a close up, partial section view of a directional sheath of a sixth alternative embodiment, within a blood vessel during a thrombectomy, the sheath including a flip-cap to access the sheath;

FIG. 23 is a close up, partial section view of the directional sheath of the sixth alternative embodiment with the flip-cap in an open mode;

FIG. 24 is a close up, partial section view of a directional sheath of a seventh alternative embodiment, the sheath within an iliac artery during a contralateral leg angiography; and

FIG. 25 is a close up, partial section view of a directional sheath of an eighth alternative embodiment, the sheath with another iliac artery during an isolated medication perfusion procedure.

### DESCRIPTION OF THE CURRENT EMBODIMENTS

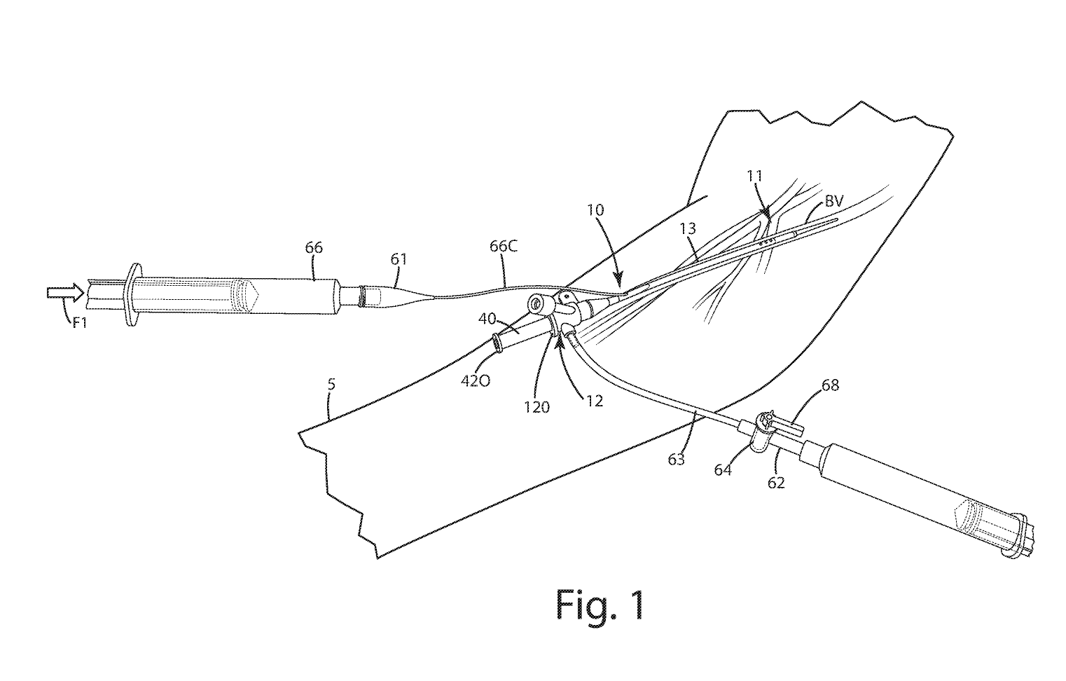

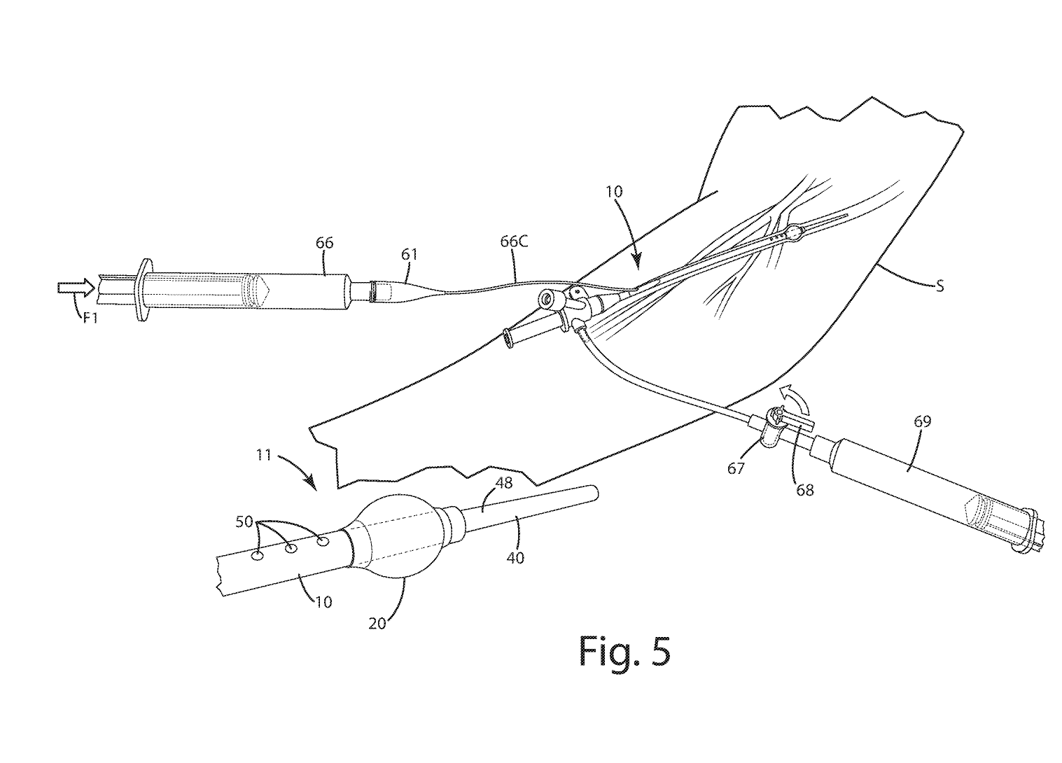

A current embodiment of the directional sheath and method of use is illustrated in FIGS. 1-12 and generally designated 10. As shown in FIG. 1, the directional sheath 10 is configured for administration of fluids to a blood vessel BV of a subject. A health care provider can manipulate the directional sheath to perform a variety of different procedures including angiography procedures, thrombectomy procedures, and optionally any other procedure that might benefit from retrograde administration of a fluid or liquid to a blood vessel.

It is to be noted that while most of the current embodiments as explained in connection with angiography, and in particular an arteriovenous fistulogram (AV fistulogram), the directional sheath herein can be used for injecting a variety of different liquids into blood vessels, optionally retrograde. Some examples of such procedures and other materials that can be administrated retrograde or antegrade via the current embodiments of the directional sheath herein include: contralateral leg angiography, isolated limb perfusion of chemotherapy, and isolated hemolytic therapy. Generally, the current embodiments of the directional sheath can be utilized for any injection of a substance, such as contrast dye, medication, blood, thrombolytic agents, thrombotic agents, stenting materials or other materials, into a subject's blood vessel during arrestment of blood flow within the blood vessel, in a retrograde or antegrade manner, with the sheath pointed in either direction of blood flow, for example, pointed upstream or pointed downstream.

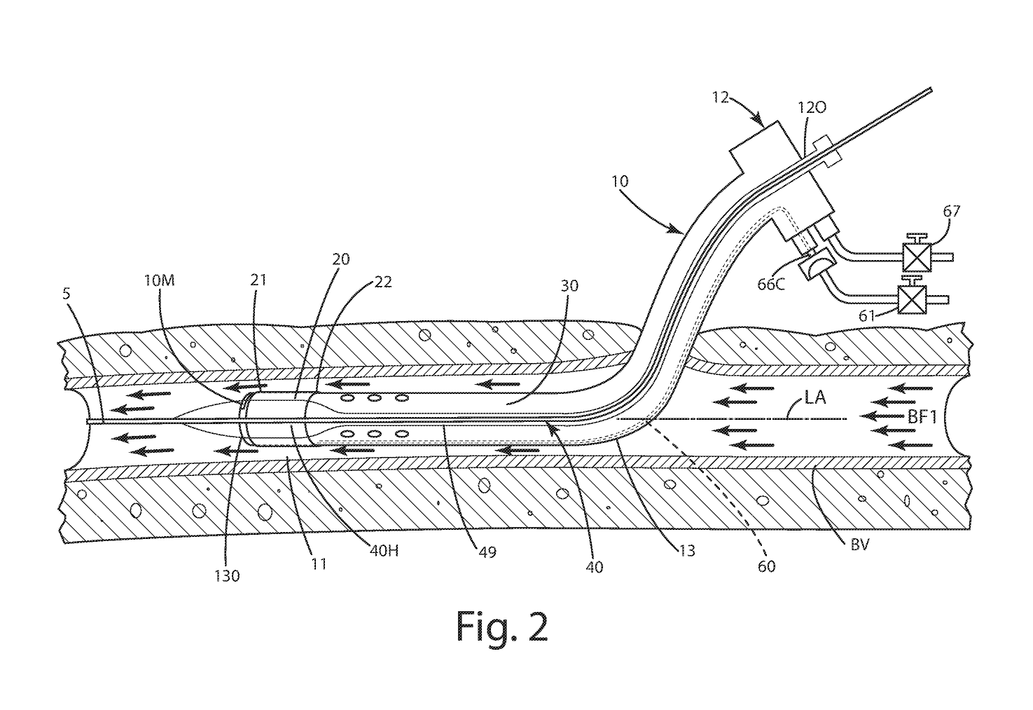

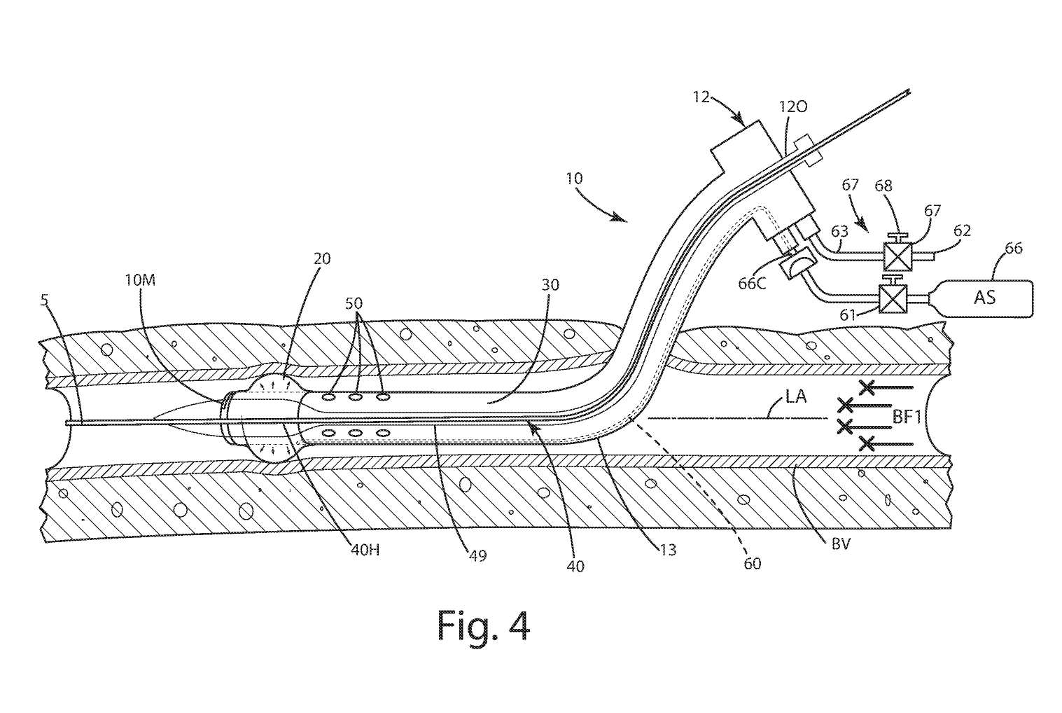

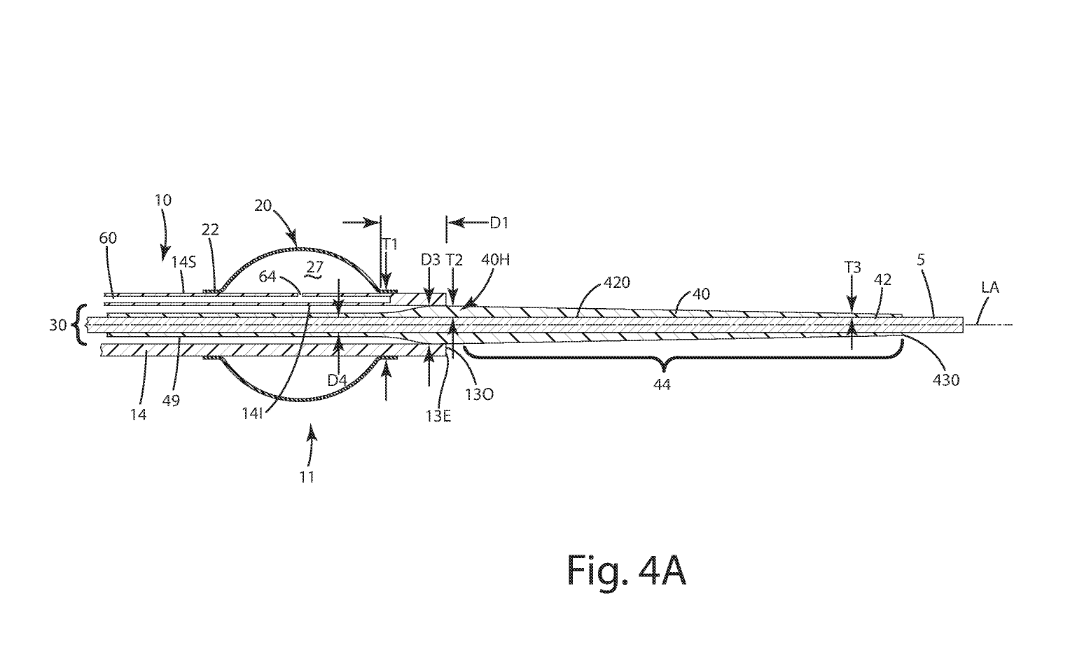

Returning to FIGS. 1 and 2, the directional sheath 10 includes a distal end 11 and a proximal end 12. These ends are distal from one another and include an intermediate sheath portion 13 therebetween. The distal end 11 generally includes a sheath opening 130 that opens to the environment. The distal end opening 1300 can be bounded by a circumferential edge 13 E of the distal end of the sheath. Although shown as an abrupt and squared off terminating portion, this edge 13E can be tapered and can extend outwardly along at least a portion of the dilator, reducing in diameter as shown in FIG. 4A. The distal end can be configured for subcutaneous insertion into the blood vessel BV, optionally facilitated via the taper noted above. This distal end adjacent the opening 130 can be slightly conical, shaped as a truncated cone, without the dilator installed. The directional sheath can be guided into the blood vessel by a guide wire 5. The guide wire 5 can be configured so that it fits within the internal guide wire lumen 42 defined by the dilator 40, and generally within the directional sheath, extending along its longitudinal axis LA. The guide wire can be significantly longer than the directional sheath and can be inserted into the blood vessel before the sheath is installed as further explained below.

The distal end 11, and the sheath 10, in general can include an occlusion balloon 20. This occlusion balloon can be constructed from an inflatable member, such as a flexible membrane, that is adapted to expand upon inflation about the distal end of the sheath 10. The occlusion balloon can be inflatable when fluid is injected into it, and collapsible when the fluid is retracted from it. Optionally, the balloon can be constructed from a very thin, airtight membrane. The occlusion balloon can completely circumferentiate the distal end of the sheath 10, so as to form a seal between the blood vessel wall and the sheath during a procedure as described herein.

Generally, the occlusion balloon can be secured directly to the exterior sheath wall 14 an in particular, the sheath exterior surface 14S, of the wall. This attachment can be via the application of an adhesive to attach the balloon. Of course, other attachments can be used, for example, the balloon can be mechanically trapped using a flange extending from the balloon and secured to the distal end of the sheath. Other structures are also contemplated for attachment of the balloon.

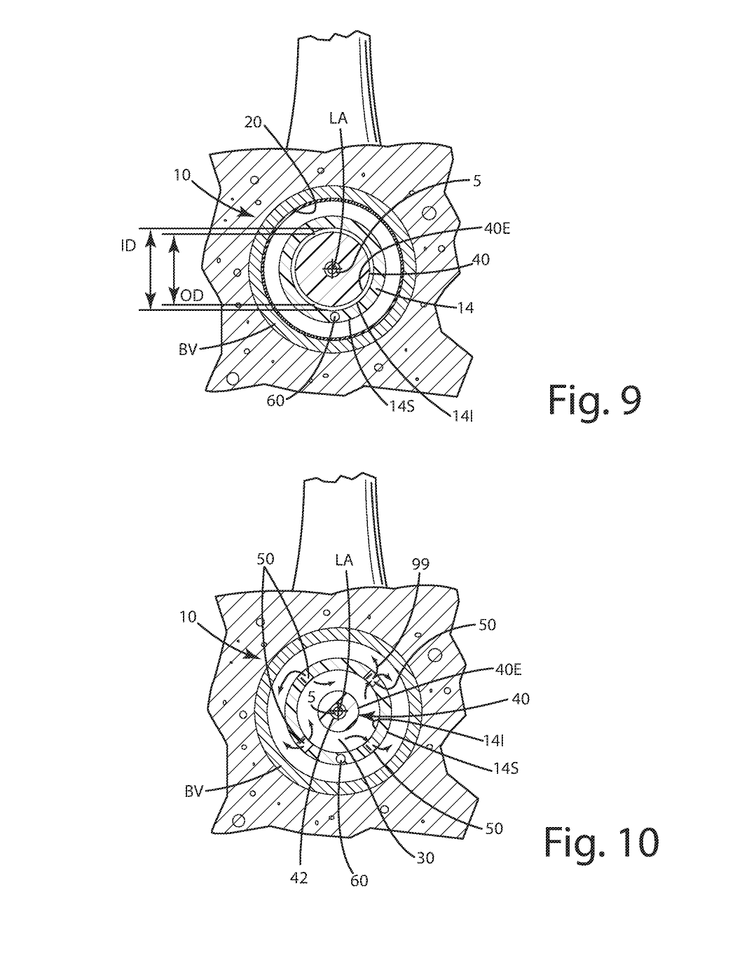

As shown in FIGS. 2-4A, the occlusion balloon 20, can be non-movable relative to the longitudinal axis LA of the directional sheath 10. It can, however, expand radially outwardly away from the longitudinal axis, generally outwardly away from the sheath exterior surface 14S (FIG. 9) again to provide the occlusion effect as explained above. The occlusion balloon 20 can include a forward end 21 and a rearward end 22 (FIG. 4A). The forward end 21 is closer to the sheath opening 130 than the rearward end 22. That distance D can be selected to optionally be about 0 mm to about 20 mm, further optionally about 1 mm to about 10 mm, even further optionally, about 1 mm to about 5 mm. Of course, other distances can be selected depending on the particular application and the blood vessel to be occluded using the occlusion balloon 20.

As mentioned above and as shown in FIGS. 1, 2 and 4A, the sheath 10 can include an internal sheath lumen 30. This internal sheath lumen 30 can generally extend from the distal end 11 toward the proximal end 12, generally forming an open cavity between those two ends. The internal sheath lumen can be of a generally circular cross section as shown in FIG. 10. Of course, the internal sheath lumen 30 can be of a variety of other cross sections depending on the particular application and other desired lumens defined within the directional sheath, for example, those described below. The internal sheath lumen 30 can be bounded by the exterior sheath wall 14. In particular, the sheath interior surface 141 can bound the internal sheath lumen 30. The exterior wall 14 defines one or more apertures 50 that extend from the exterior surface 140 to the interior surface 141 of the sheath exterior wall 14. The sheath exterior surface 140 is open to the environment, for example, the blood and other liquids flowing around the sheath 10 when placed in a blood vessel. The sheath interior surface 141 generally faces toward the dilator 40, and in particular the sheath exterior surface 40E. The sheath interior surface 141 and the dilator exterior surface 40S form an internal fluid passageway 52 therebetween, which is used to convey fluid, such as contract dye or other fluids described herein to the apertures 50 defined by the exterior sheath wall 14.

Optionally, the internal sheath lumen includes an inner diameter ID that is larger than the dilator outer diameter OD as shown in FIG. 9. Generally, the dilator is specially formed and configured to have varying dimensions along its length as described further below to create the internal fluid passageway 52 within the sheath 10 and in particular the exterior sheath wall 14.

For example, the sheath interior surface 141 faces toward the dilator 40 as explained above, and as shown in FIG. 9. The dilator exterior surface 40E contacts the sheath interior surface 141 where the dilator is of an enlarged dimension, for example, at the distal end 11 of the sheath 10. This contrasts other portions of the sheath, for example, the sheath intermediate portion 13, which again is located between the distal end and the proximal end. In this location, the sheath interior surface 141 is spaced a distance from the dilator 40, and in particular the dilator exterior surface 40E due to a reduced dimension in the dilator. This, in turn forms the internal fluid passageway 52 within the internal sheath lumen 30.

As shown in FIGS. 2-10, the intermediate portion 13 of the sheath defines apertures 50. These apertures 50 are generally disposed rearward of the distal end opening 130 and forward of the proximal end opening 120. Generally, these apertures do not form any portion of the sheath through which another structural element of the sheath 10 projects. In most cases, the only material conveyed through the apertures 50 is fluid that is within the internal fluid passageway 52.

The apertures 50 as illustrated are in the form of circular holes linearly disposed along the sheath, and extending through the exterior sheath wall, optionally defined along a line parallel to the longitudinal axis LA. Of course, these apertures can be of different shapes, for example, they can be slots, curved apertures, rounded apertures, elongated apertures, etc. Further, the apertures 50 can be in a helical configuration, spiraling around the longitudinal axis LA of the sheath 10 on the exterior sheath wall 14. In some cases, the apertures can be in the form of elongated slits that extend rearwardly, away from the distal end 11 of the sheath. Further, although explained in most embodiments herein as being multiple apertures, a single enlarged aperture can be substituted for multiple smaller ones. However, such a single aperture can be prone to plugging or occlusion via a portion of a blood vessel, which is why multiple apertures can be better suited for some applications. Further, the apertures are disposed generally distal from the opening 130 of the distal end 11. Between the apertures and the distal end opening 130, the occlusion balloon 20 is disposed. This is so the desired fluid can be injected into the blood vessel via the apertures 50, rearward of the occlusion balloon 20.

Optionally, the sheath can include a radio-opaque marker 10M (FIG. 1), which can be viewed via fluoroscopy, X-ray or other imaging procedures. The marker 10M can be placed adjacent the distal end 11, or can extend along the length of the sheath. For example, adjacent the opening 130 of the sheath with the marker, the healthcare provider can precisely identify the location of the end of the sheath within the blood vessel using imaging techniques. This can enable the healthcare provider to more accurately position the sheath and the apertures for the applicable procedure.

As shown in FIGS. 4A, 9 and 10, the sheath 10 can define an inflation lumen 60. This inflation lumen, although referred to in connection with inflation, can also be used for deflation, relative to the occlusion balloon 20, or other conveyance of fluid within the lumen 60. The inflation lumen 60 can be offset from the longitudinal axis LA of the sheath. Further, it can be non-coaxial with the internal sheath lumen 30 if desired. The inflation lumen 60 can extend from the distal end 11, adjacent the occlusion balloon 20 to the proximal end 12 of the sheath 10. The inflation lumen can be in communication with an inflation port 61 via a connector conduit 66C. The inflation port 61 can be configured to join with a syringe or other fluid supply to convey fluid into and through the inflation lumen selectively.

As shown in FIG. 4A, the inflation lumen 60 can include an aperture 64 that extends from the inflation lumen 60 to the exterior surface 14S of the sheath wall 14 so that fluid may be conveyed through that aperture 64, into the occlusion balloon 20. The inflation aperture 64 can be a single aperture, or can be multiple apertures defined between the inflation lumen 60 and the interior cavity 27 of the occlusion balloon 20. In most applications, the inflation lumen can be configured to convey a fluid, such as oxygen or air, into the occlusion balloon. In some cases, however, the fluid can be a liquid to provide particular inflation performance of the occlusion balloon 20.

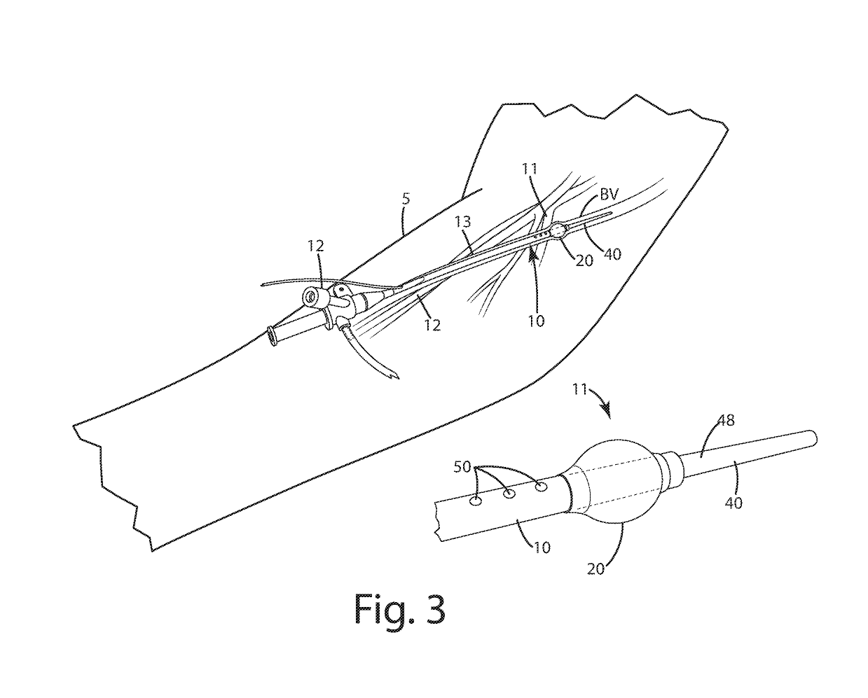

As shown in FIGS. 3-4A, the inflation lumen 60 extends as mentioned above from the distal end 11 to the proximal end 12. At the proximal end, the port 61 can be connected to a source of fluid 66, which as illustrated is in the form of a syringe. The syringe can be manually operated by depressing the plunger to expel fluid, such as oxygen, from the syringe into the conduit 66C which is in fluid communication with the inflation lumen 60. Alternatively, there can be a separate port or other connection to provide fluid communication between the inflation syringe 66, or some other air source and the inflation lumen.

As shown in FIGS. 9-10, the inflation lumen 60 can be a separate lumen defined within the directional sheath 10, separated from the internal sheath lumen 30 and located somewhere within the exterior sheath wall 14 and/or within the sheath exterior surface 14S. Optionally, the inflation lumen is separated from the apertures 50 a sufficient distance so as not to interfere with the operation of those apertures, and still provide fluid flow to the occlusion balloon 20. The inflation lumen also can include an inflation lumen longitudinal axis, which can be separate, distal and/or offset from the longitudinal axis LA of the internal sheath lumen 30 and/or any optional secondary lumens.

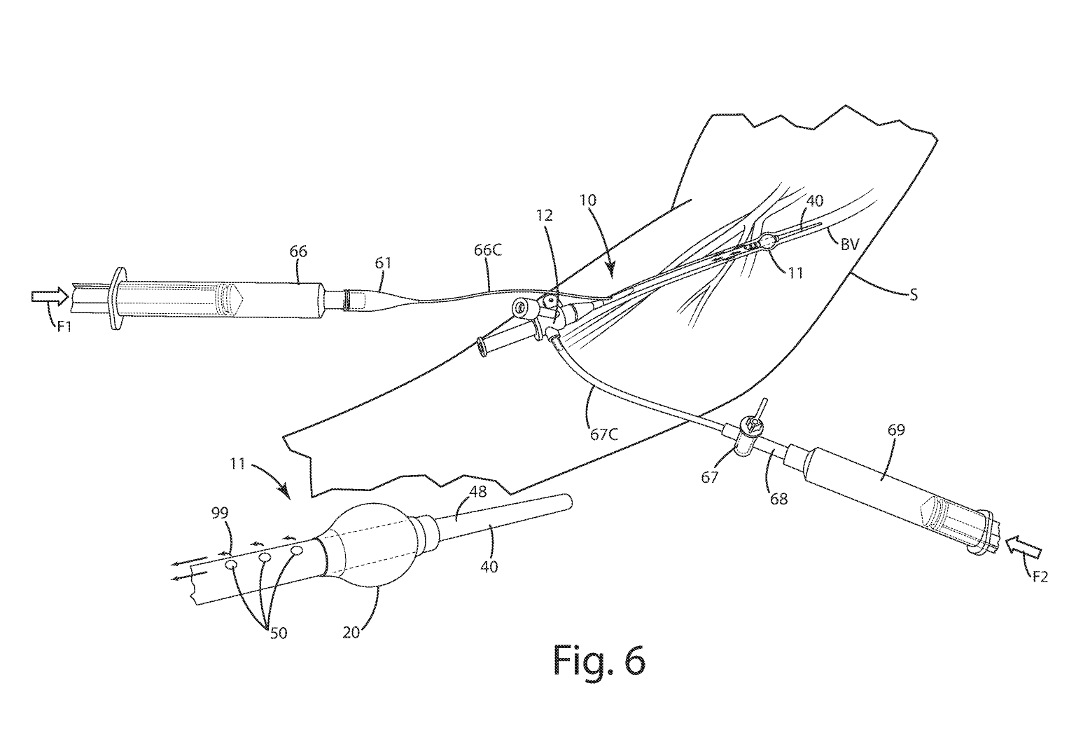

As mentioned above, and as shown in FIGS. 2 and 6, the proximal end 12 of the sheath can include the inflation port 61 and a secondary port 67, also referred to herein as an injection port. The injection port 67 can be outfitted to receive a syringe 69 or other device. This syringe or other device can be configured to inject fluid, such as liquid, into the secondary port 67 and subsequently into the internal sheath lumen 30. Optionally, the syringe can be filled with a contrast dye, such as fluorescein, useful in the angiography procedures herein. Other types of contrast dyes or dyes suitable for an angiography can be utilized in its place.

As shown in FIGS. 1 and 2, the distal end 12 of the directional sheath can include a terminal end opening 120. This opening 120 can include a one-way valve or other penetrable membrane. The opening 120 can generally receive a guide wire 5 extending through it and the sheath, the end opening 120 also can be of sufficient size and structure to enable the dilator 40 to be installed through it. In addition, as mentioned further below, the dilator 40 can include an opening 420 through which the guide wire 5 can extend.

Optionally, the sheath and its respective components and the dilator can be of varying lengths. For use with small blood vessels or readily accessible blood vessels, the sheath and dilator can be optionally 2 inches to 12 inches long, further optionally about 6 inches to 10 inches long. For use with large blood vessels or inaccessible blood vessels, the sheath and dilator can be optionally 12 inches to 36 inches long, further optionally about 18 inches to 24 inches long.

As shown in FIGS. 1, 2, 4A, 9 and 10, the dilator 40 generally is an elongated element with a distal end 42 and a proximal end 41. The proximal end 42 is located again adjacent the proximal end 12 of the sheath 10 when fully installed and in use. The distal end 42 is partially disposed within the tip of the sheath, at the distal end 11. A portion 44 of the dilator 40 however, extends beyond the end opening 130 of the distal end 11 of the sheath. This portion 44 of the dilator can be of a tapered configuration with its sidewalls diminishing toward a dilator end opening 430 at the distal end 42 of the dilator. The walls of the dilator can taper from a greater thickness T2 to a lesser thickness T3, as shown in FIG. 4A. These thicknesses can vary depending on the desired taper and the blood vessel within which the sheath is used.

Adjacent the distal end 11 of the sheath and/or the opening 130, the dilator can have an enlarged tip or head 40H, which is of dimension D3. This enlarged dimension D3 is sized so that the exterior surface of the dilator can engage the interior sheath surface 141 of the internal sheath lumen 30 and occlude the distal end of the sheath so that fluid and/or liquid does not readily seep past the dilator 40, through the opening 130 of the sheath when installed as illustrated in FIG. 4A. The dilator 40 can further include a second, lesser or reduced dimension D4. This dimension D4 is lesser than the dimension D3 of the enlarged head 40H of the dilator. As an example, the dimension D3 can be a first diameter and the dimension D4 can be a second diameter. The second diameter can be less than the first diameter. With this construction, the fluid passageway 52 of the sheath can be established between the reduced dimension D4 of the dilator and the interior surface 141 of the sheath wall 14.

As illustrated in FIG. 4A, the dilator can include a varying wall thickness, as mentioned above. For example, near the opening 430 of the dilator, the wall thickness can be a thickness T3. This thickness T3 can increase to thickness T2 adjacent the opening or portion of the dilator adapted to engage the internal sheath lumen. The dilator wall thickness can further vary starting rearward of the exposed portion 44 of the dilator distal end 42. For example, the dilator wall can include a thickness T1 in the region adjacent the occlusion balloon 20 when the dilator 40 is inserted in the sheath 10. This thickness can be approximately equal to the thickness T3, and generally less than the thickness T2 of the dilator wall adjacent the opening 130 of the sheath, in the enlarged head 40H of the dilator. The dilator wall also can taper in a rounded and/or abrupt manner when transitioning from the wall having a thickness T1 to the wall having a thickness T2. As illustrated from there, toward the tip, the dilator wall can taper again in the opposite way, becoming thinner until ultimately thinning to the thickness T3 adjacent the opening 430. This thinning of the wall can correspond directly with the regions of the reduced diameter D4 of the dilator and the increased diameter D3 of the dilator. That is, the dilator wall thickness can be the greater thickness T2 and at the increased dimension D3 of the dilator. The lesser thickness T1 can be associated with the reduced dimension D4, which is less than the dimension D3 of the dilator.

Optionally, the dilator can include an enlarged head 40H, mentioned above, having a greater diameter adjacent dilator the distal end than in the remainder of the dilator body extending rearwardly therefrom. Further optionally, the dilator distal end includes the enlarged head 40H which is joined with a thinner body portion 49 of the dilator as illustrated in FIG. 4A. The enlarged head 40H can have a sufficient outer dimension or diameter D3 to effectively occlude the end of the sheath opening 130. In contrast, the body portion 49 of the dilator body 49 is of an insufficient dimension D4 to completely occlude the end opening 130 of the sheath 10.

Further optionally, the sheath distal end 11 can include a detent or other protrusion that provides tactile feedback to a user when the enlarged head 40H is sufficiently inserted and occluding the end of the sheath. Alternatively, the dilator can be of a specific length so that when fully inserted into the sheath, it bottoms out and cannot be inserted any farther.

The dilator, as mentioned above can define an internal guide wire lumen 42 within which the guide wire 5 can be disposed. The guide wire lumen 42 as illustrated in FIG. 9 can generally by coaxial with the longitudinal axis of the directional sheath 10. Optionally, the lumen 42 can be centered in the cross section of the sheath.

The various components of the sheath and the dilator can be constructed from a variety of polymeric materials. These materials can be coated with coatings, such as Teflon® or other lubricious materials, to assist in insertion into a blood vessel.

A method of using the directional sheath of the current embodiment will now be described in further detail. In this method, an angiography procedure is conducted, in which an angiography dye is introduced into a blood vessel to facilitate imaging of the blood vessel and related structure such as fistula. The dye as described in this method can be a dye such a fluorescein. Further, it should be noted that although described in connection with conducting an angiography, the directional sheath used herein can be utilized in the introduction of any type of fluid or liquid into a blood vessel through the sheath.

As described below, the method specifically can be used in conjunction with an AV fistulogram to evaluate a fistula upstream from the insertion point of the directional sheath. Generally, the directional sheath can administer the angiography dye retrograde, that is, against the normal blood flow BF1 within the blood vessel, as shown in FIGS. 1-7. To begin the method, an angiography needle (not shown) is inserted into the blood vessel BV. A guide wire 5 is then inserted into the needle, and it subsequently enters the blood vessel. With the guide wire sufficiently disposed in the blood vessel, the needle is removed. The sheath and dilator are then aligned with the guide wire. The guide wire 5 is inserted into the guide wire lumen 42 defined by the dilator 40. The sheath 10 and dilator 40 are slid as a unit along the guide wire 5, through the tissue of the subject S and into the blood vessel BV as shown in FIGS. 1 and 2. The guide wire 5 extends out the terminal end or opening 120 of the directional sheath 10, along with the dilator. The distal end 11 of the sheath, with the enlarged dilator head 40H are disposed generally within the blood vessel BV as shown in FIG. 2. Blood continues to flow in direction of blood flow BF1, as shown in FIG. 2. The sheath 10 and dilator 40 are generally pointed in the same direction as the blood flow BF1. Of course, in other applications, the sheath and dilator can be inserted so that the tip points upstream in a direction opposite to that of blood flow BF1 shown in FIGS. 1-4.

Returning to FIG. 2, the sheath can include a marker 10M at its distal end. This marker can be visualized by a healthcare provider viewing an X-ray scan of the subject's blood vessel at the insertion site. The provider can use this information and place the proximal end 11 precisely where desired within the blood vessel BV.

With the distal end of the sheath properly positioned in the blood vessel, a user can attach an inflation syringe 66 to the inflation port 62 and/or inflation conduit 66C which is further in communication with the inflation lumen 60. The user can depress or apply a force on the plunger of the inflation syringe 66. This, in turn, pushes fluid, such as oxygen or air out from the syringe, through the inflation lumen 60. The fluid is conveyed through the inflation lumen 60, through the inflation aperture 64, into the internal cavity 27 of the occlusion balloon 20. This inflates the balloon 20 causing it to expand radially outward from the longitudinal axis LA of the sheath. In turn, the outer surfaces of the balloon engage the inner surfaces of the blood vessel BV. The balloon can substantially circumferentiate the distal end 11 of the sheath thereby closing off the blood vessel BV. In some cases, as shown in FIG. 4., the balloon's inflation can alter the shape of the blood vessel BV, causing it to bow slightly outwardly. The occlusion balloon can be specifically matched with the particular blood vessel within which it is to be disposed to insure it does not rupture the blood vessel, yet still properly occludes it for the procedures herein.

The inflated occlusion balloon 20, shown in FIGS. 4-6, occludes the blood vessel. With this action, the blood flow BF1 can stop as shown in FIG. 4 in the direction that it was previously flowing. A healthcare provider can attach a syringe 69 to the inflation port or secondary port 67. The syringe can be filled with a contrast dye, or other fluid desired to be administered to the interior of the blood vessel. The user can actuate a manual control or lever 68 of the port 67, rotating it in the direction as shown with the arrow. This can open a valve within the port 67. The user can apply a force F2, as shown in FIG. 6, to the syringe, thereby dispelling out from it the angiography dye 99. The dye 99 is conveyed through the conduit 67C and into the sheath. The dye as shown in FIG. 10 continues to flow through the fluid passageway 52 defined between the interior surface or inner diameter of the internal sheath lumen 30 and the exterior diameter, or exterior surface 40E of the dilator 40. Again, because of the reduced dimension of the dilator, this internal fluid passageway exists.

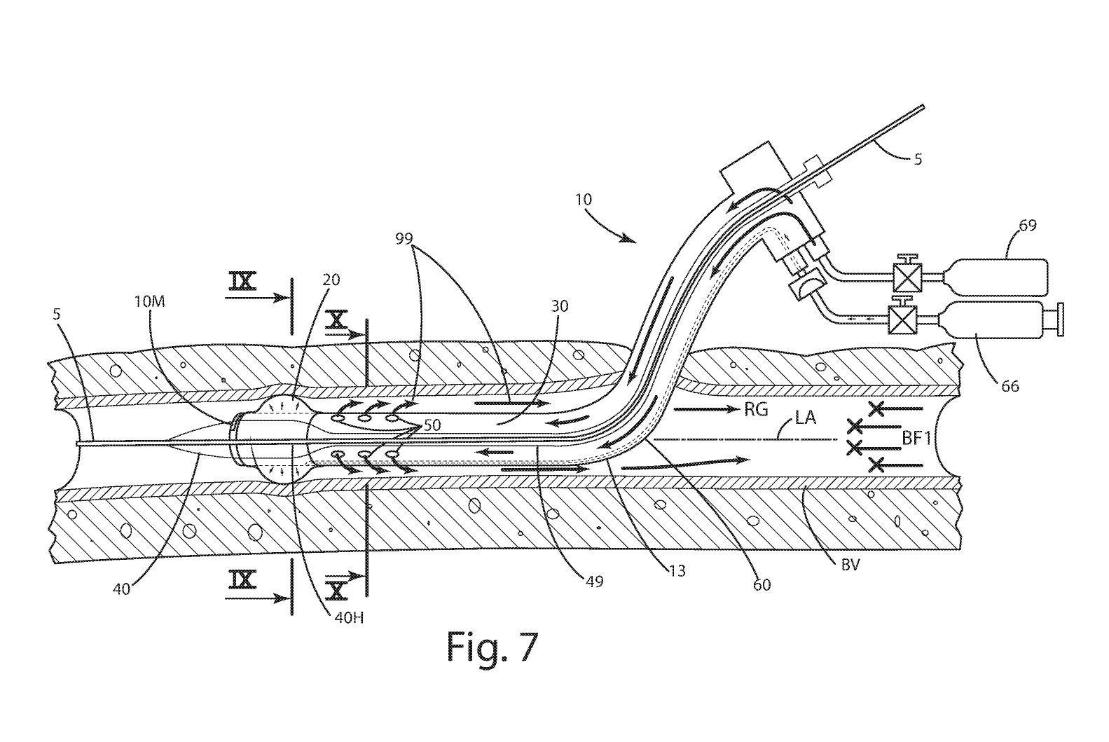

The fluid passageway 52 is in fluid communication with the apertures 50 defined by the sheath wall 14. Therefore, the angiography dye 99 injected via the syringe 69 under pressure continues to flow, ultimately exiting those apertures 50 into the environment surrounding the sheath, within the blood vessel. This is illustrated in the inset of FIG. 6. The force F2 continues to be applied. The dye 99 continues to flow out the apertures 50 and flows retrograde RG (FIG. 7) into the blood vessel BV, counter or against the previous blood flow BF1 that was flowing through the blood vessel. Typically, a sufficient amount of angiography dye 99 is injected into the blood vessel BV so that the dye can travel upstream to a fistula formed with the blood vessel BV and beyond. This can enable the healthcare provider to view the fistula and the vein to insure integrity and/or observe any stenosis.

During the above process, with the enlarged tip 40H of a dilator occluding the end of the sheath or the sheath opening 130, the angiography dye 99 does not exit that opening and travel downstream of the sheath distal end 11. Further, due to the occlusion of the balloon 20 in the blood vessel BV, that dye 99 does not seep substantially past the balloon. Thus, the blood vessel BV is effectively “plugged” using the sheath, dilator and occlusion balloon to prevent the dye 99 from traveling downstream of its insertion point. The angiography dye 99 does not flow downstream with the blood flow within the blood vessel. Further, the dye does not flow out the end opening of a sheath.

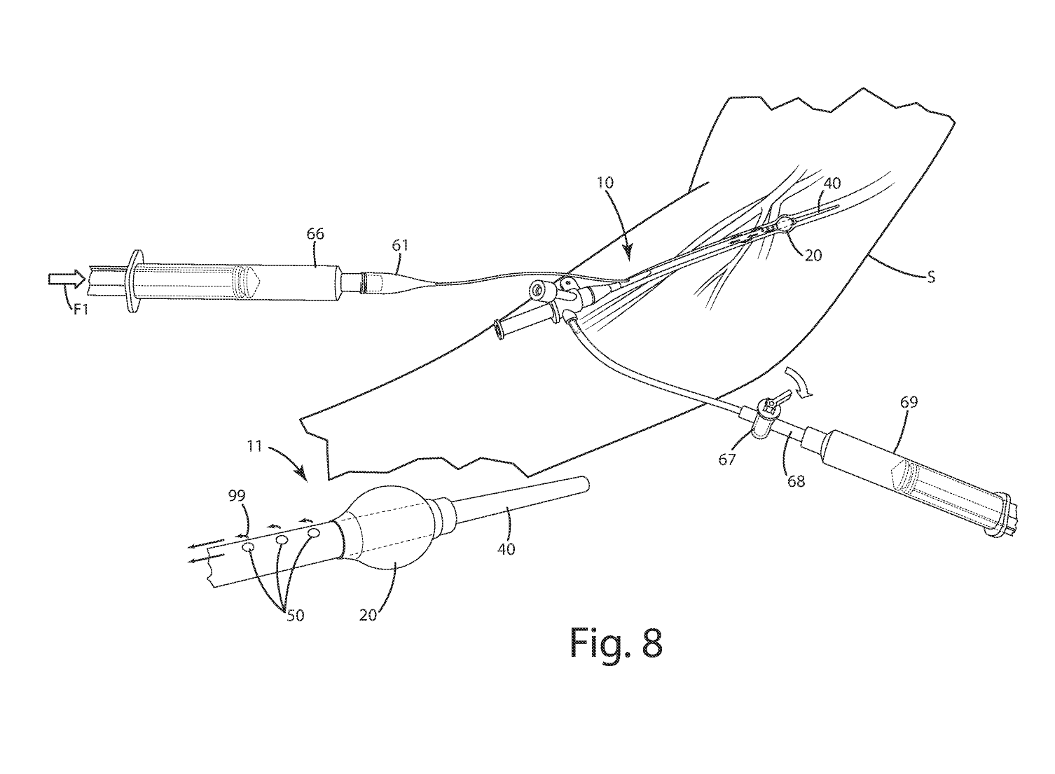

When a sufficient amount of angiography dye 99 has been injected into the blood vessel as shown in FIG. 8, a healthcare provider can take an X-ray image of the subject's arm and blood vessel BV. In doing so, and due to advantages of the current embodiments, the provider need not hold their own hand under the X-ray device, subjecting it to radiation. Again, with the current embodiments, there is no need to hold any portion of the blood vessel or otherwise manually occlude the blood vessel when administering the dye retrograde. After a sufficient amount of imaging, the secondary port 67 is closed by rotating the control 68 in the direction. No more force at that time is applied to the syringe 69.

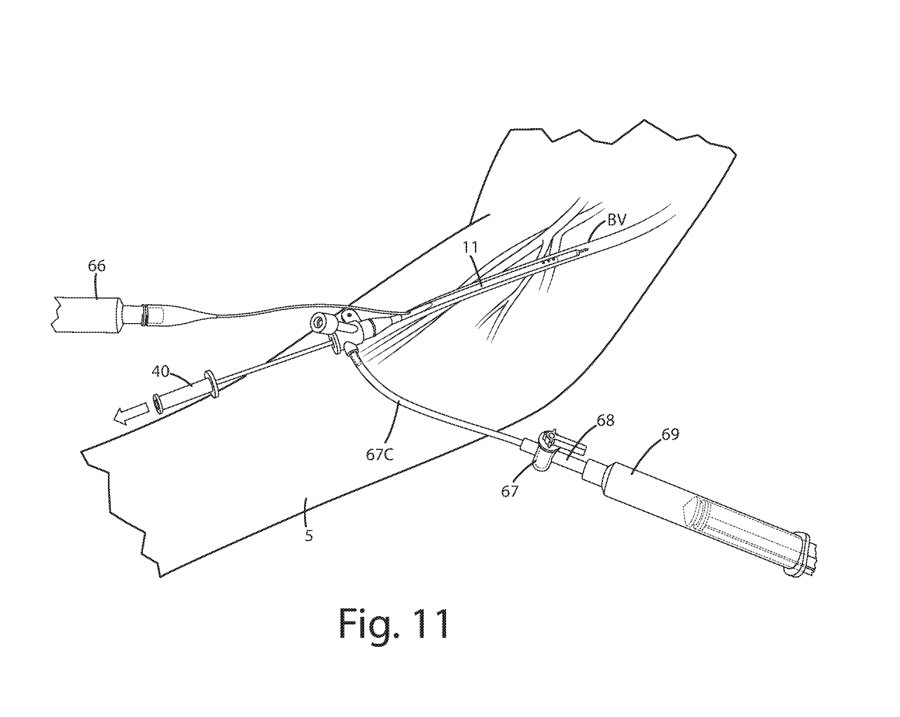

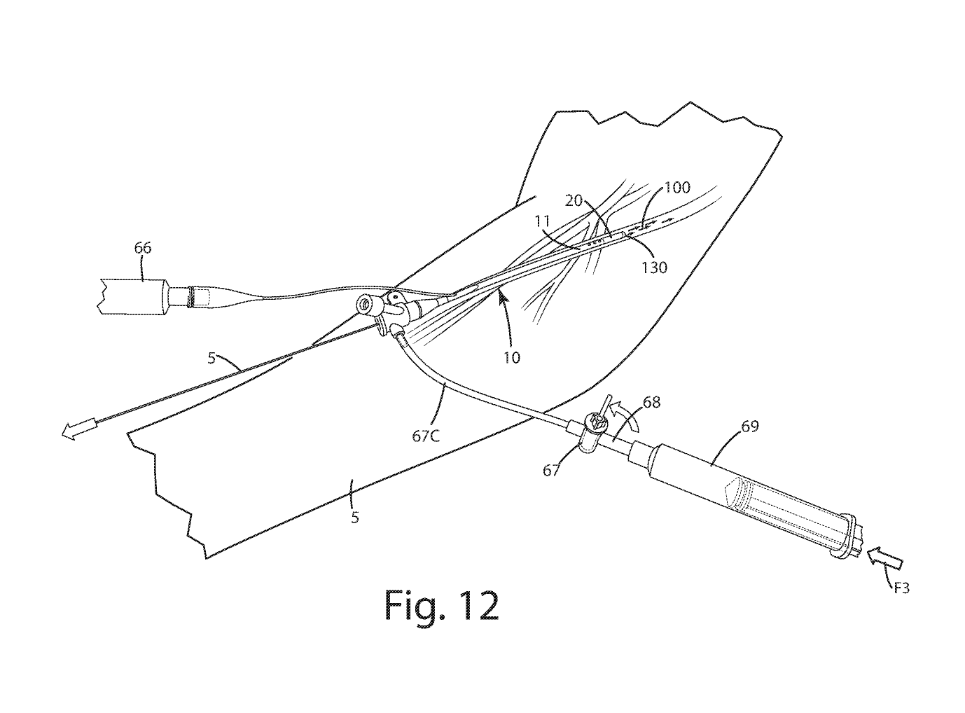

If desired, the healthcare provider can administer the dye 99 downstream, to take a further angiogram of the blood vessel structure downstream of the sheath insertion point. To do so, as shown in FIG. 11, the provider removes the dilator 40 from the sheath. The occlusion balloon 20 optionally can be deflated, or it can be left inflated if desired to arrest blood flow in its normal direction of flow within the blood vessel. As shown in FIG. 12, the provider then manipulates the control 68 to open the port 67 and inject additional dye by applying a force F3 to the syringe 69. The dye flows through the conduit 67C and into the internal sheath lumen 30 of the sheath. Because the dilator 40 has been removed from the sheath 10, the contrast dye 99 travels out the opening 130 of the distal end 13. This angiography dye 100 flows downstream in the direction of the blood flow BF1. This can push and carry the dye with the blood flow traveling past the distal end 11 of the sheath. As the contrast dye is administered, another X-ray image of the blood vessel BV and the respective dye can be taken to evaluate the integrity of the blood vessel and look for any stenosis. Optionally, after the angiography procedure, the sheath can be removed along with the guide wire.

Further optionally, where the occlusion balloon remains inflated, and the dilator is removed from the sheath, the sheath can be used to administer a substance other than dye while the occlusion balloon restrains and/or arrests the blood flow within the blood vessel. As an example, the sheath can be used to introduce a medication, drug, chemotherapy, thrombolytic agent, thrombotic agent, blood product (such as blood, plasma, red blood cells, etc.) and or stent material directly into the blood vessel, antegrade, in the direction of blood flow, while the balloon maintains an inflated state, thereby occluding the blood vessel, and restraining blood flow through it. If desired, the sheath distal end and occlusion balloon can be inserted into a blood vessel facing upstream, against normal blood flow, or into a blood vessel facing downstream, with normal blood flow within the blood vessel.

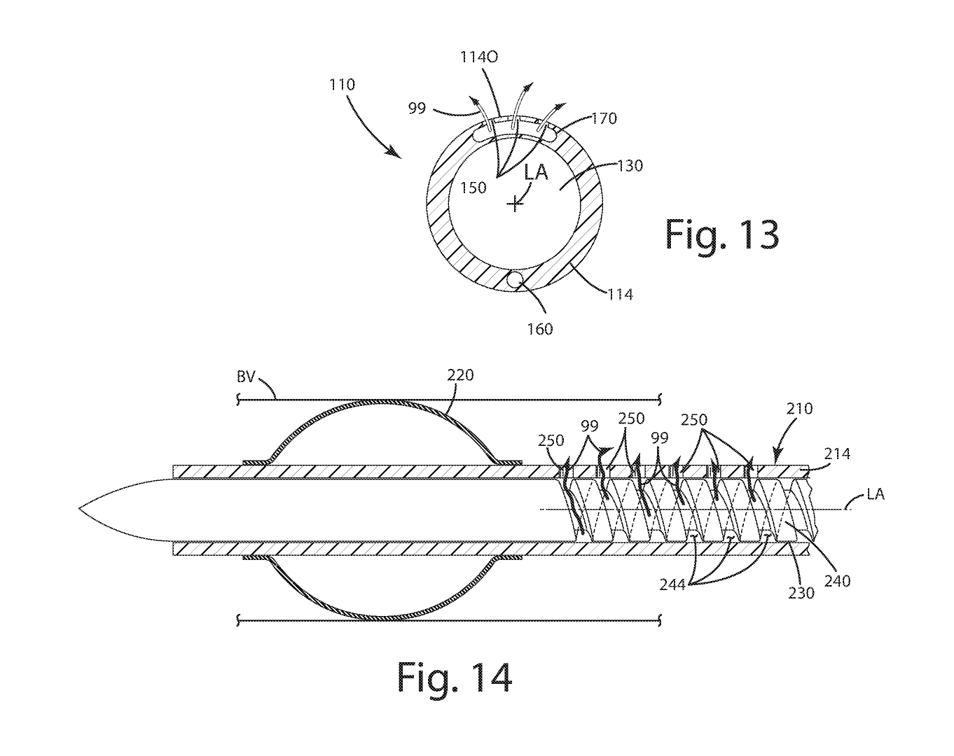

A first alternative embodiment of the directional sheath is illustrated in FIG. 13 and designated by the reference numeral 110. This embodiment can be similar in structure, function and operation to the embodiment described above with several exceptions. For example, the sheath 110 defines an internal sheath lumen 130 that extends along a substantial portion of the sheath. The sheath 110 also includes a sheath wall 114 which defines the internal sheath lumen 130. Within this sheath wall 114, an inflation lumen 160 can be defined, separate from the internal sheath lumen 130. This inflation lumen 160 can be in communication with an occlusion balloon similar to that described in the embodiment above. The inflation lumen 130 can be sized to receive a dilator similar to that in the embodiment above, however, the dilator can be of a uniform diameter throughout its length.

In this embodiment, the sheath wall 114 can define a secondary lumen 170 which also can be referred to as an injection lumen or a contrast lumen. This injection lumen can be defined directly and entirely in the wall of the sheath 110, distal and separate from the inflation lumen 160. Alternatively, it can be defined adjacent the sheath wall, next to the internal sheath lumen. Generally, both the inflation lumen and injection lumen can be offset from the longitudinal axis LA of the sheath 110. The injection lumen 170 can be of a circular, elliptical, polygonal or other cross section. As illustrated, the lumen 170 can be elongated, lumen spanning along the top side of the sheath 110, generally diametrically opposed to the inflation lumen 160 across the longitudinal axis LA. The injection lumen 170 also can define apertures 150 that open to the environment, and through the sheath exterior surface 114S. These apertures 150 can enable a contrast dye or other fluid 99 to exit the injection lumen 170 directly into the environment surrounding the sheath 110, which can be a blood vessel as with the embodiment above. If desired, there may be multiple additional injection lumens 170 extending along the directional sheath 110, generally defined within the sheath wall 114.

A second alternative embodiment of the directional sheath is illustrated in FIG. 14 and generally designated 210. This second alternative embodiment can be similar in structure, function and operation to the embodiments above with several exceptions. For example, a plurality of apertures 250 are defined in the outer sheath wall 214. The sheath can include an occlusion balloon 220 designed to engage an inner diameter of the blood vessel and occlude the blood vessel in the region adjacent the enlarged tip 242 of the dilator 240. The dilator 240, however, can define a recess, groove, aperture or other void 244, extending in a helical fashion around the exterior of the dilator, generally along the longitudinal axis. This groove can be in fluid communication with the plurality of apertures 250 when the dilator is installed within the internal sheath lumen 230 of the sheath 210, particularly when a contrast dye or other fluid is injected into the internal sheath lumen 230. The injected fluid can flow around the helical path ultimately out the apertures 250 and into the blood vessel BV. The width, depth, pitch, and angle of the helical groove can be varied, depending on the particular application.

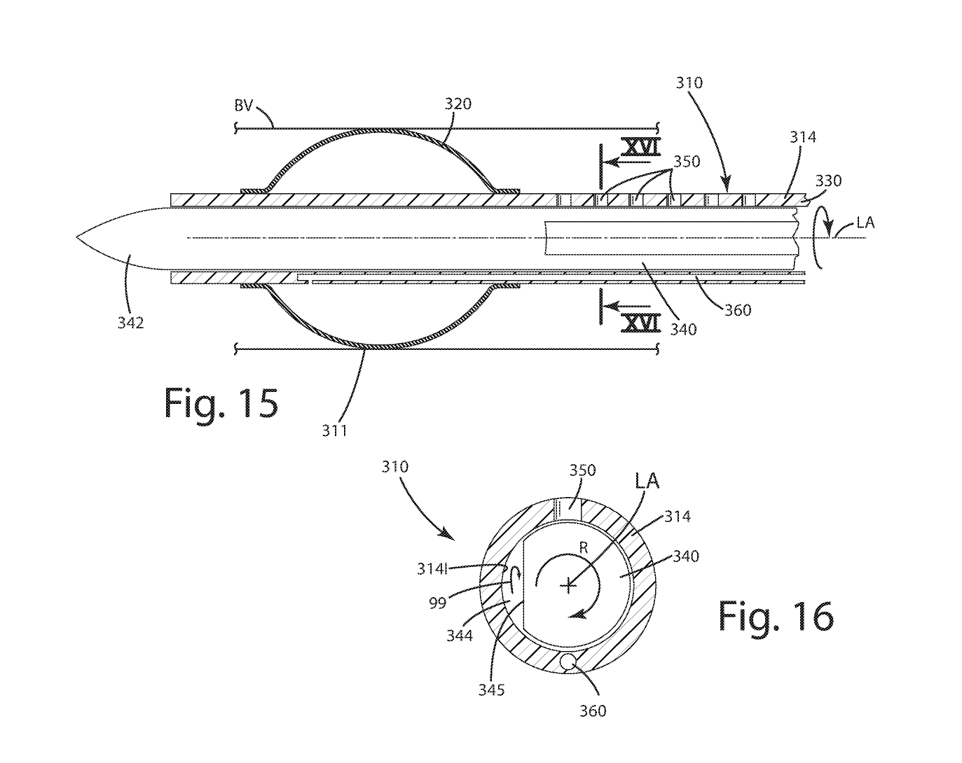

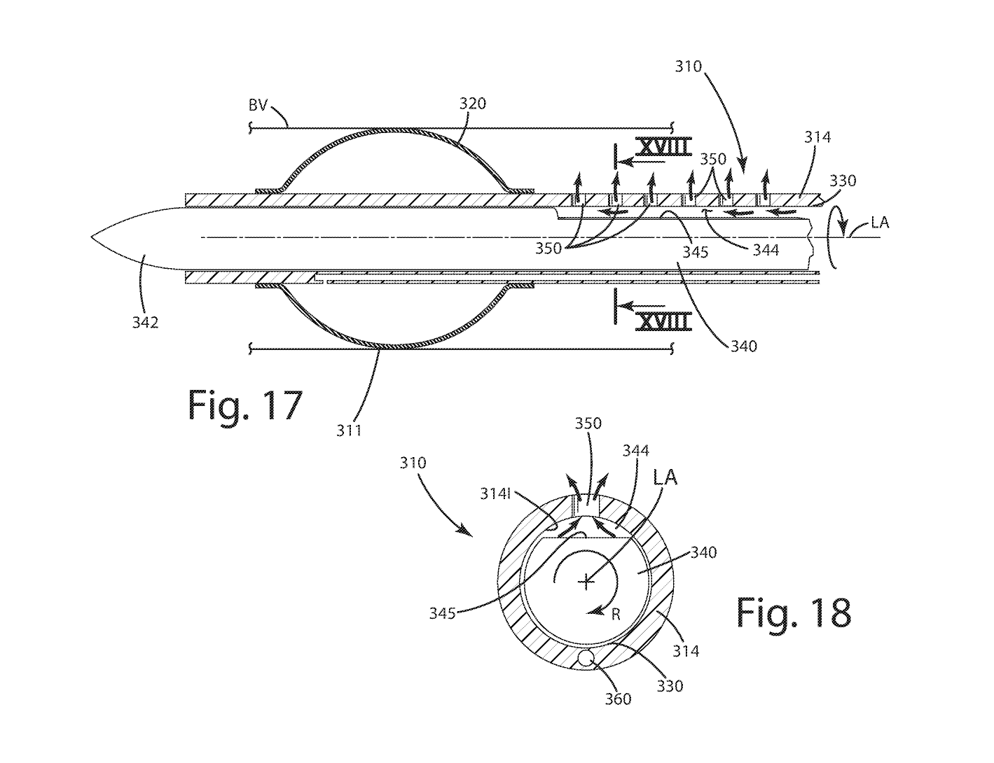

A third alternative embodiment of the directional sheath is illustrated in FIGS. 15-19 and generally designated 310. This embodiment can be similar to the embodiments above in structure, function and operation, with several exceptions. For example, the sheath 310 includes a dilator 340 disposed therein and an occlusion balloon 320 disposed at the distal end 311 of the sheath. The occlusion balloon 320 can be in fluid communication with an inflation lumen 360 similar to that described in the embodiments above. The sheath defines an internal sheath lumen 330 which is bounded generally by an exterior sheath wall 314. The exterior sheath wall defines apertures 350 similar to those above. In this embodiment, however, the dilator 342 can generally be of a constant exterior dimension which is within a close tolerance of the inner diameter or dimension of the internal sheath lumen 330. The dilator can be configured to include a recess, groove, cut-out or other passageway 344 adjacent one of its exterior surfaces. As an example, the dilator can include a removed portion in the form of a chord. With this chord removed, or simply not present when formed, the dilator includes an outer relief surface 345. Between this outer relief surface 345 and the interior sheath surface 3141, the passageway 344 is defined as shown in FIG. 16. This can enable contrast dye to flow alongside the dilator 340, within the fluid passageway 344 defined between the relief surface 345 and the interior surface 3141 of the sheath wall 314, generally toward the apertures 350. When the dilator is positioned as shown in FIG. 16, the contrast dye 99 will not flow out from the apertures 350 defined by the sheath wall 314 because there is not fluid communication between those elements and the passageway 344. To actuate the dilator and thus the sheath, enabling fluid to flow out from the apertures 350 as shown in FIG. 18, a provider rotates the dilator 340 in direction R. This reconfigures the dilator relief surface 345 so that it generally faces toward and/or is aligned with the apertures 350. Thus, the fluid passageway 344 defined between that surface and the interior surface 3141 of the sheath 310 is in fluid communication with the apertures 350. The dye 99 can flow through the passageway 344 and out the apertures 350. The application of the contrast fluid can be similar to that described in conjunction with the embodiments above, utilizing an injection syringe and the respective ports and valves.

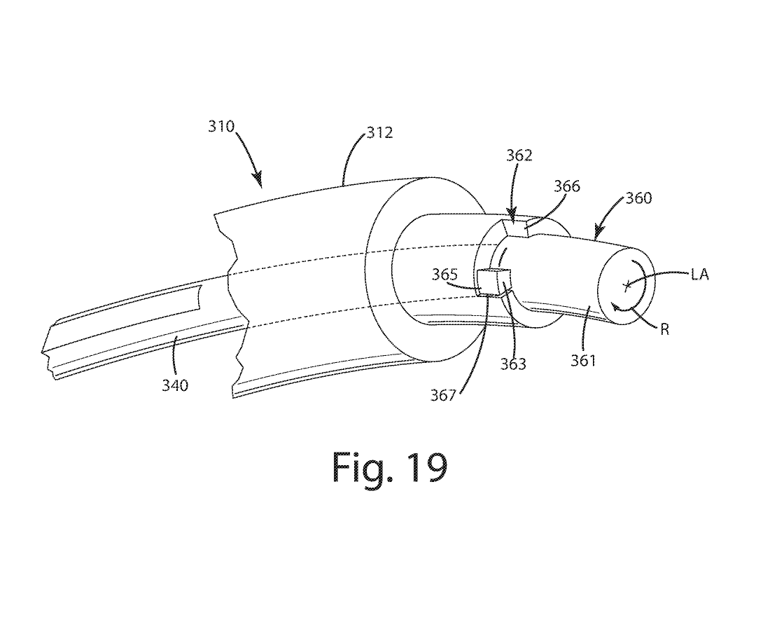

As shown in FIG. 16, rotation R of the dilator 340, can be about the longitudinal axis LA. This rotation can be controlled with an adjuster 360 as shown in FIG. 19. The adjuster 360, can be in the form of a manually operable projection or protrusion extending from a proximal end 312 of the directional sheath 310. The manual control 360 can be directly attached to the dilator 340 extending toward the distal tip of the sheath. By rotating the adjuster 360 in the direction R, it subsequently rotates the dilator 340 in the same direction.

In many cases it is suitable to precisely rotate the dilator 340 so that the dilator relief surface 345 and passageway 344 is in direct alignment with the apertures 350. To provide this type of alignment, the adjustor 360 can be outfitted with an indexer or limiter 362. This indexer can include a projection or plate 363 extending from a manually rotatable knob 361. The projection 363 can be registered in a recess 365. The recess can include a stop or limit wall 366.

When the rotatable knob 361 is rotated in the direction R, that rotation is limited when the projection 363 engages the stop or limit wall 366. Upon this engagement, the fluid passageway 344 is aligned with the apertures 350 as shown in FIG. 18. Reversing this direction of rotation, so that the projection 363 engages the other wall 367, the dilator occludes the apertures 350 as shown in FIG. 16 and the passageway 344 is no longer in fluid communication with those apertures. Of course, other adjusters can be utilized to rotate the dilator to provide the desired effect.

Optionally, the adjuster can facilitate selective rotation of the dilator in any increment. For example, the indexer or limiter 362 can be configured to allow selective rotation of optionally 45° to 180°, further optionally about 45° to 90°, or other varying amounts of rotation. Further optionally, when fully rotated, the limiter 362 can audibly click or provide a visual indication so that the provider can confirm that the dilator has been appropriately moved.

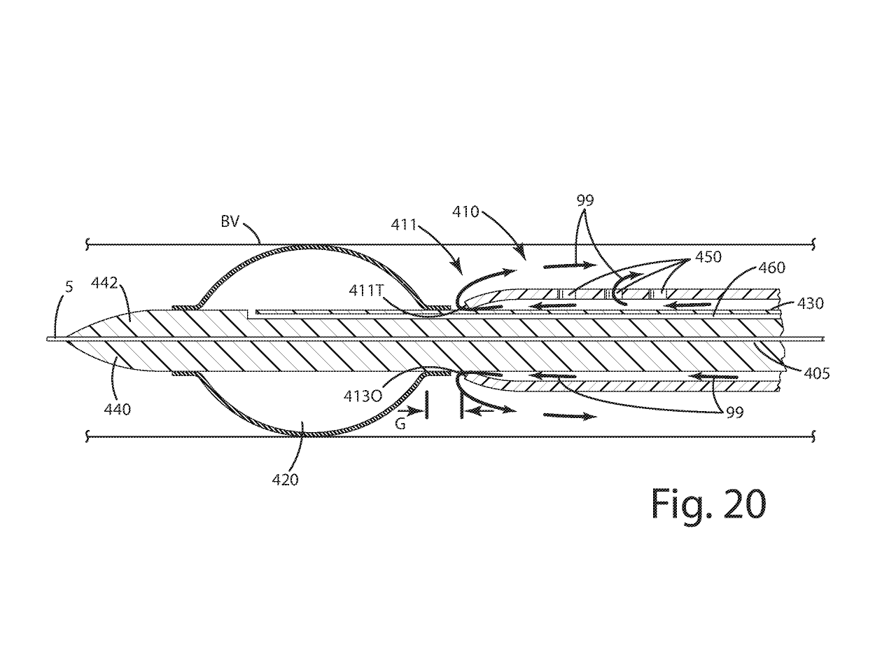

A fourth alternative embodiment of the directional sheath is illustrated in FIG. 20 and generally designated 410. This embodiment can be similar in structure, function and operation to the embodiment described above with several exceptions. For example, the directional sheath includes a dilator 440 disposed in an internal sheath lumen 430. The internal sheath lumen 430 is bounded by an exterior sheath wall 414 which defines apertures 450 near the distal end 411 of the sheath. The distal end 411 of the sheath includes a tip or outermost portion 411T which defines a sheath distal end opening 4130. The dilator 440 extends beyond this opening 4130 and includes a tapered tip 442. Adjacent the tip 442, an occlusion balloon 420 is joined directly with the dilator 440, optionally using a structure similar to those used to connect the occlusion balloon to the sheath as described in the embodiments above.

The dilator in this embodiment also defines at least one internal inflation lumen 460 which can be similar to the inflation lumen in the directional sheath above, except that the inflation lumen is defined in the dilator, not the sheath. Of course, this inflation lumen 360 can be separate and distal from a guide wire lumen 405 through which a guide wire 5 can be disposed. Indeed, the axes of both these lumens can be offset from one another. Optionally, the occlusion balloon 420 can be configured so that when the dilator is fully inserted into the sheath 440, a gap G is formed between the occlusion balloon 420 and the opening 4130 and/or tip 411T of the sheath. The dilator occlusion balloon 420 can be configured to fully occlude the blood vessel BV.

To administer a contrast dye or other fluid out the sheath and optionally retrograde, the contrast dye is first introduced into the internal sheath lumen 430 as described in the embodiments above. The dye 99 travels toward the distal end 411. Where included, it can exit through the sheath wall directly through the apertures 450 and out into the blood vessel BV. Further optionally, the dye 99 can exit out the tip 411T, through the opening 4130 directly beside the dilator. In some cases, the walls of the sheath adjacent the tip 411T can be flexible, and can bend outward, allowing additional fluid to pass thereby and into the blood vessel. Because the occlusion balloon 420 on the dilator is spaced the gap G from the tip, it does not substantially impair the flow of the dye from the tip into the blood vessel. Further, with the occlusion balloon 420 occluding the blood vessel, the injected dye does not flow past that occlusion balloon, downstream of the sheath 410.

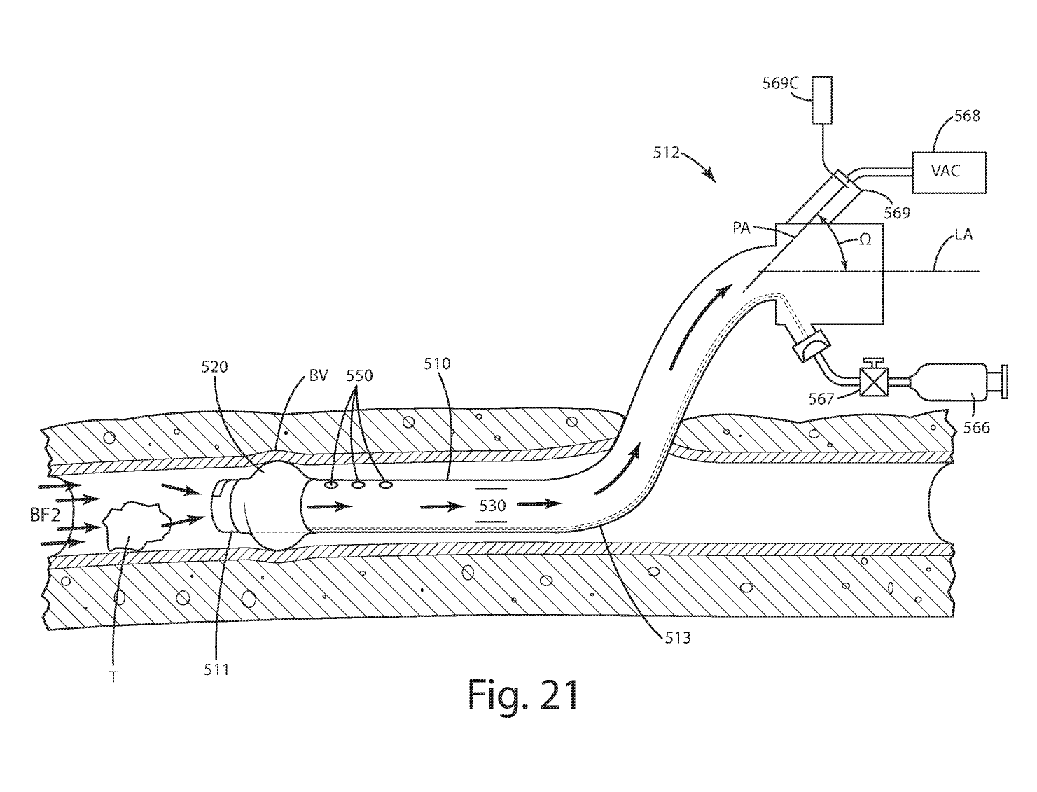

A fifth alternative embodiment of the directional sheath is illustrated in FIG. 21 and generally designated 510. This embodiment can be identical in structure, function and operation to the embodiments described above with several exceptions. For example, the sheath 510 can include all the different components including the distal end 511, intermediate portion 513 and proximal end 512 of the sheath 510 as with the other embodiments, and also can define an internal lumen 530 and respective ports 569 and 567. The sheath can include an occlusion balloon 520 that is adapted to engage a blood vessel BV wall to occlude the blood vessel. The sheath also can define the apertures 550 in its sidewall.

This embodiment, however, is for use in a thrombectomy. In particular, a healthcare provider may identify a thrombus T, also referred to as a blood clot, within a particular blood vessel BV. The provider can insert a sheath needle and guide wire into the blood vessel BV downstream (or upstream, if desired) of the thrombus T. The directional sheath, with a dilator (not shown) similar to that in the embodiments above, can be guided on the guide wire into the blood vessel BV as explained in the embodiments above. The occlusion balloon 520 can be inflated to engage the blood vessel wall, generally circumferentiating the blood vessel and arresting normal blood flow. The dilator (not shown) can be removed. At this point, the sheath is generally downstream of the blood flow BF2 and the thrombus T. A user can apply a vacuum or negative pressure 568 through the port 569. This in turn will suck fluid or liquid into the distal end 511 of the sheath. With this negative pressure, the thrombus T can be dislodged and pulled into the directional sheath for removal. The vacuum can be turned off, and the occlusion balloon can be deflated. Thereafter, the directional sheath can be removed, with the thrombus T therein, thereby removing the thrombus from the blood vessel BV.

Optionally, the port 569 can be of a tubular structure and selectively closed by a cap 569C (shown removed). The cap 569C can be a flip top cap or a screw on cap so that the port can be readily accessed for use in a thrombectomy or other procedure. As shown, the port 569 and its axis PA can extend transversely from the proximal end 512 of the sheath. The port can be offset at an angle 1 from the longitudinal axis LA of the sheath, so that the port 569 does not interfere with the operation of a dilator. The angle 1 can be optionally about 5 degrees to about 45 degrees, further optionally about 10 degrees to about 30 degrees, depending on the application and tools desired to be place through the port.

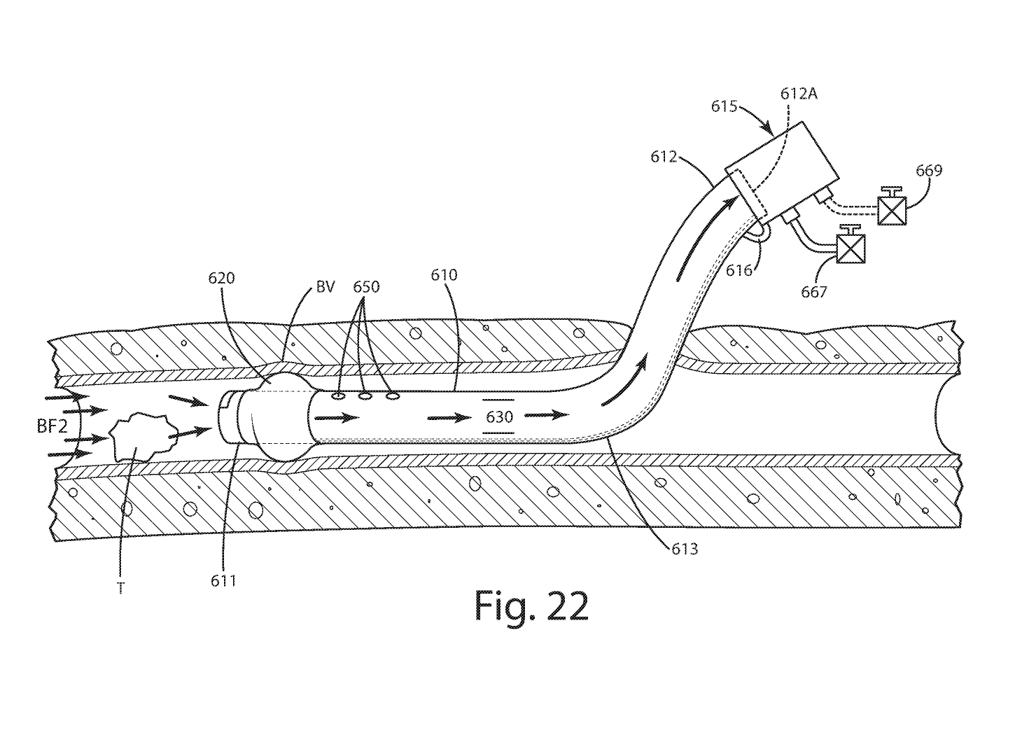

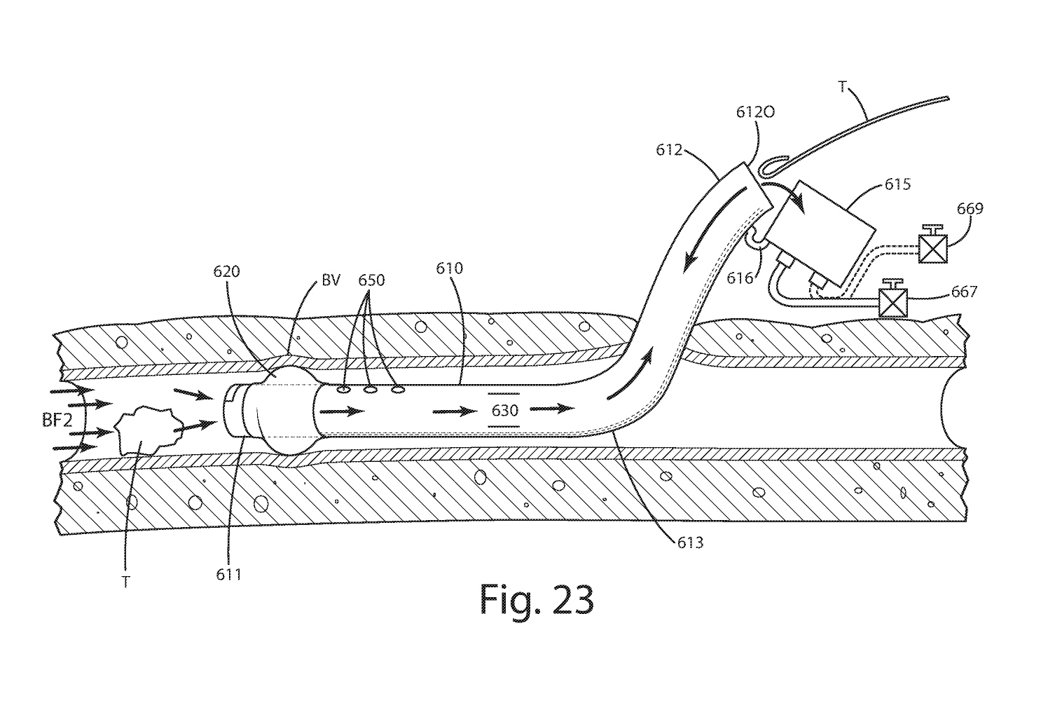

A sixth alternative embodiment of the directional sheath is illustrated in FIGS. 22 and 23 and generally designated 610. This embodiment can be identical in structure, function and operation to the embodiments described above with several exceptions. For example, the sheath 610 can include all the different components including the distal end 611, the intermediate portion 613, proximal end 612 of the sheath 610 as with the other embodiments, and also can define an internal lumen 630 and respective inflation and injection ports 667 and 669 which are adjacent the distal end 612. The sheath can include an occlusion balloon 620 that is adapted to engage a blood vessel BV wall to occlude the blood vessel and arrest or impair normal blood flow. This sheath also can define the apertures 650 in its sidewall.

This embodiment, however, also can be used in connection with a thrombectomy, and optionally used to insert a tool T into a blood vessel to retrieve or remove a thrombus T. For example, the sheath 610 can be outfitted with an end piece or end cap 615. As illustrated in FIG. 22, the end piece 615 can be removably attached or joined with the proximal end 612 of the sheath. An end portion 612A of the proximal end projects into the end piece 615 as shown in broken lines FIG. 22. The end piece 615 can be hingeably attached to the distal end 612 via a flexible, pivoting and/or bendable hinge 616. In this construction, the end piece can function as a flip cap, modifiable between open and closed modes. When the port 667 is in use, the end piece 615 can be in communication and connectedly joined with the distal end 612 as shown in closed mode configuration in FIG. 22. However, when it is desired to access the internal lumen 630, the end piece 615 can be flipped open like a “flip cap” as shown in FIG. 23. In this case, it moves in the direction of the arrow to open the proximal end 612 so that the opening 6120 of the proximal end 612 is exposed to the environment. The port 667 can also remain attached to and in communication with the end piece 615. With the opening 6120 exposed, a tool T can be inserted into the internal lumen and can project therethrough, optionally, out the distal end 611 to access the thrombus T. Alternatively, a suction unit (not shown) can be generally attached to the proximal end 612 to apply a negative pressure to pull the thrombus from the blood vessel.

Further optionally, instead of the illustrated hinged construction, the end piece 615 can be releasable from the proximal end 612 of the sheath 610 using other constructions. For example, the proximal end portion 612A can be threaded in an internal bore and the end cap 615 can be likewise threaded onto that portion. When a user desires to remove the end piece 615, the user can simply unthread it from the proximal end 612. In another construction, the end piece 615 can simply be friction fit into the proximal end 612, without any additional hinges or threading. Other constructions are contemplated for releaseably attaching the end cap 615 to the proximal end 612 of the sheath 610.

With the above construction and the removable end piece 615, a healthcare provider can remove a thrombus T from a blood vessel, and leave the sheath inside the blood vessel. The provider can replace the end piece. After it is believed that the thrombus T has been adequately removed, the provider can utilize the port 667 to inject a contrast dye into the internal sheath lumen 630 and out the distal end 611 of the sheath. The provider can then attain an x-ray image and view the contrast dye within the blood vessel to determine whether or not the thrombus has been adequately removed.

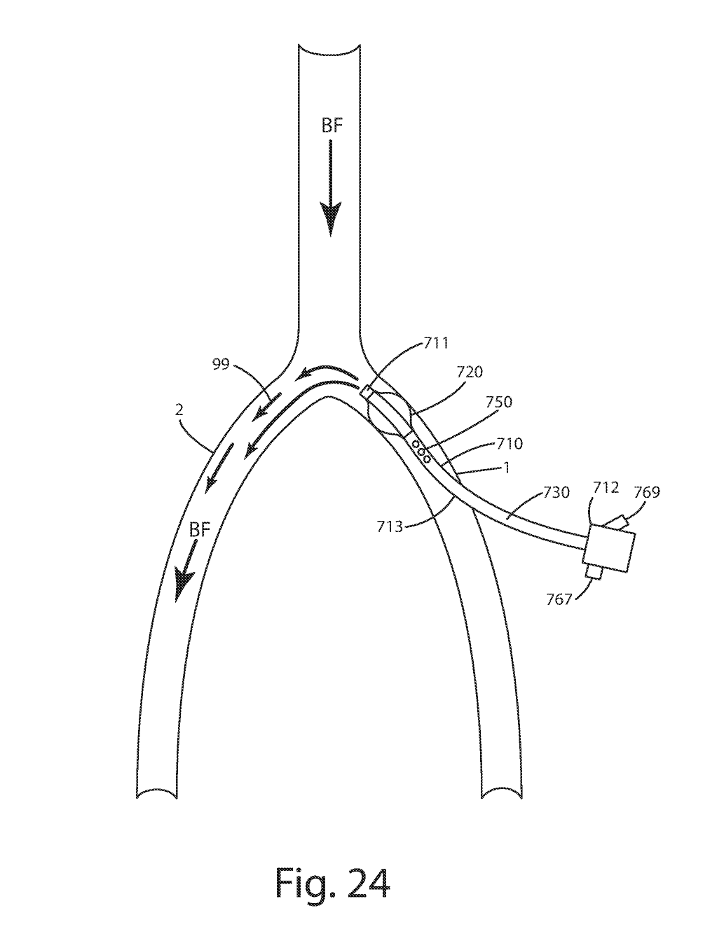

A seventh alternative embodiment of the directional sheath is illustrated in FIG. 24 and generally designated 710. This embodiment can be identical in structure, function and operation to the embodiments described above with several exceptions. For example, the sheath 710 can include all the different components including the distal end 711, intermediate portion 713 and proximal end 712 of the sheath 710 as with the other embodiments, and also can define an internal lumen 730 and respective ports, for example the inflation port 767 and an injection port 769. The sheath can include an occlusion balloon 720 that is adapted to engage a blood vessel wall, for example the side walls of an iliac artery, to occlude the first iliac artery. The sheath also optionally can define apertures 750 in its sidewall.

This embodiment, however, can be used in conjunction with a contralateral leg angiography procedure. In particular, a healthcare provider may desire to perform such a procedure. Access to the first iliac artery 1, for example shown on the right side of FIG. 24, can be easier due to its proximity to the groin. The second iliac artery 2 on the left of FIG. 24 can be more difficult to access due to its proximity to the abdomen. If the second iliac artery is the one upon which an angiography is to be performed, with the directional sheath of this embodiment, this can be greatly facilitated. For example, the healthcare provider can insert the directional sheath 730 into the first iliac artery 1 as shown in FIG. 24. The user can remove a dilator after insertion (not shown). The port 767 can be used to inflate the occlusion balloon 720 to close off or arrest blood flow into the first iliac artery. Upon inflation of the occlusion balloon 720, the first iliac artery is closed off. Blood flow BF continues in the direction of the arrows as illustrated in FIG. 24. The provider may then join a contrast dye syringe (not shown) to the port 769 and inject contrast dye into the internal lumen 730. The contrast dye travels through the internal lumen and out the distal end 711 until the dye 99 ejects into the second iliac artery, traveling with the blood flow BF in the same. The provider can then take an x-ray or other image of the second iliac artery. This can be recorded, and the healthcare provider can determine whether the blood flow within the second iliac artery is satisfactory, and/or whether there are any abnormalities in the same. Optionally in some cases, there can be apertures 750 defined by the sheath wall. Some of the angiographic dye 99 can seep through the apertures, but generally will not cause a significant issue with subsequent imaging.

In this embodiment, the occlusion balloon 720 operates to arrest or impair blood flow down the first iliac artery. Given its position, however, it does not impair blood flow to continue through the second iliac artery.

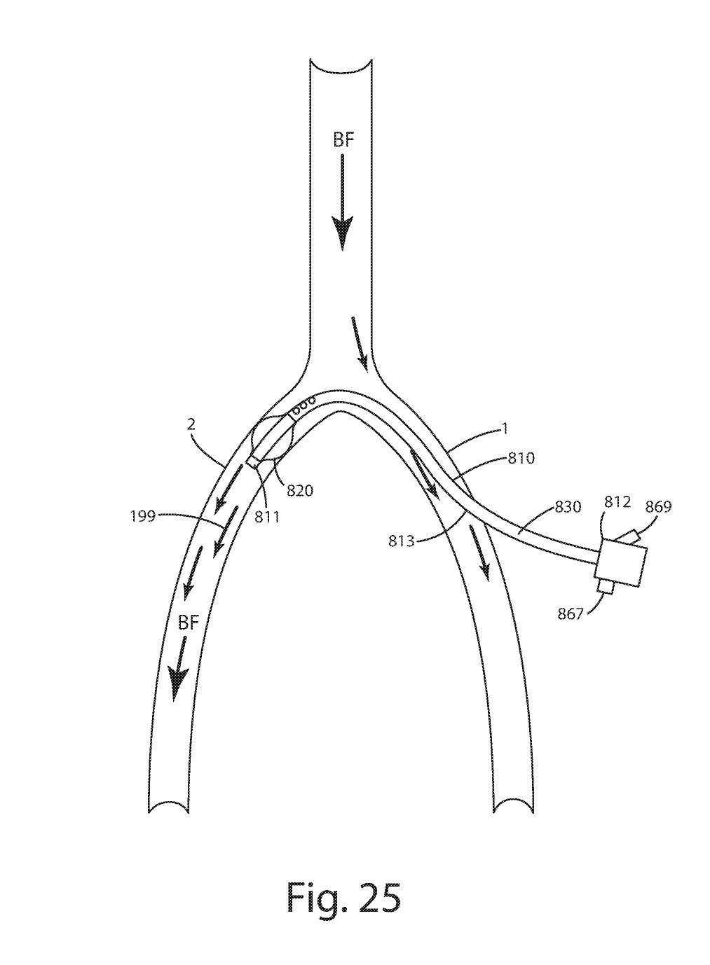

An eighth alternative embodiment of the directional sheath is illustrated in FIG. 25 and generally designated 810. This embodiment can be identical in structure, function and operation to the embodiments described above with several exceptions. For example, the sheath 810 can include all the different components including the distal end 811, intermediate portion 813 and proximal end 812 of the sheath 810 as with the other embodiments, and also can define an internal lumen 830 and respective ports 867 and 869. The sheath can include an occlusion balloon 820 that is adapted to engage a blood vessel, which as illustrated is second iliac artery that is in fluid communication with the aorta. This embodiment, however, can be used during isolated perfusion of medication into the second iliac artery 2 only. The healthcare provider can insert the sheath 810 into the first iliac artery 1 associated with the groin as illustrated to the right of FIG. 25. The healthcare provider can then convey the sheath upper end, past the aorta and down the opposing, opposite second iliac artery 2. The healthcare provider can inflate the occlusion balloon 820 as with the embodiments above. This, in turn, occludes the illustrated second iliac artery. The healthcare provider can then inject a substance 199, such as medication, drugs or chemotherapy through the port 869. This substance 199 traverses through the sheath 830 and out the distal end 811. In this manner, the substance 199 can be distributed to only the second iliac artery 2 as shown. Where the substance is a custom tailored agent or a chemotherapy agent, this can prevent the substance from substantially entering the aorta or the other opposing first iliac artery 1. As the occlusion balloon occludes the iliac artery as shown, the blood flow is redirected primarily to the second iliac artery 2, and generally arrested in the first iliac artery 1 in which the balloon is inflated. Although illustrated as administering the substance 199 antegrade, with the blood flow, where apertures are defined by a sheath (not shown), the substance can also be administered selectively in the first iliac artery 1 illustrated to the right of FIG. 25. A variety of options for injection of different substances is provided with the directional sheath. Other procedures are contemplated herein where the sheath can be used to occlude or restrict or arrest blood flow within a blood vessel in a certain direction, and then inject a substance entegrade or retrograde within the blood vessel.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular. Any reference to claim elements as “at least one of X, Y and Z” is meant to include any one of X, Y or Z individually, and any combination of X, Y and Z, for example, X, Y, Z; X, Y; X, Z; and Y, Z.

The embodiments of the invention in which an exclusive property or privilege is claimed are defined as follows:

1. A method of performing an angiography procedure comprising:
providing an introducer sheath including a sheath distal end and a sheath proximal end, the introducer sheath defining an internal sheath lumen bounded by an exterior sheath wall, the exterior sheath wall including a sheath exterior surface and a sheath interior surface, the internal sheath lumen being in fluid communication with a contrast fluid port adjacent the proximal end, the exterior sheath wall defining a plurality of apertures, the introducer sheath including an occlusion balloon located adjacent the sheath distal end;
providing a dilator including a dilator distal end and a dilator proximal end, with a dilator intermediate portion located therebetween, the dilator distal end having a first dimension, the dilator intermediate portion having a second dimension, less than the first dimension, the dilator being located within the internal sheath lumen so that the dilator distal end occludes the internal sheath lumen;
inserting the sheath distal end into a blood vessel of a subject;
inflating the occlusion balloon so that the balloon occludes the blood vessel; and
introducing an angiography dye through the contrast fluid port so the contrast dye flows into the internal sheath lumen, between the dilator intermediate portion and the sheath interior surface;
wherein the angiography dye exits the introducer sheath through the plurality of apertures so that the angiography dye is introduced retrograde in the blood vessel.

2. The method of claim 1 comprising providing an inflation lumen adjacent the introducer sheath lumen and conveying fluid through the inflation lumen to the occlusion balloon during said inflating step.

3. The method of claim 1 comprising inserting a guide wire into the blood vessel before said inserting step, wherein the guide wire guides the introducer sheath into the blood vessel during said inserting step.

4. The method of claim 1 wherein the sheath proximal end includes a balloon port, comprising conveying a fluid from the balloon port toward the occlusion balloon.

5. The method of claim 1 comprising maintaining the dilator distal end in engagement with the introducer sheath to occlude the sheath distal end during the introducing step so that no angiography dye exits the sheath distal end.

6. A method of performing an angiography procedure comprising:
providing an introducer sheath including a sheath distal end and a sheath proximal end, the introducer sheath defining a plurality of apertures between the sheath distal end and the sheath proximal end, the introducer sheath including an occlusion balloon located adjacent the sheath distal end;
providing a dilator including a dilator distal end and a dilator proximal end, with a dilator intermediate portion located therebetween, the dilator positioned so that the dilator distal end occludes the sheath distal end;
inserting the sheath distal end into a blood vessel of a subject;
inflating the occlusion balloon so that the balloon occludes the blood vessel; and
introducing an angiography dye into the introducer sheath at the proximal end so that the angiography dye exits the introducer sheath through the plurality of apertures,
whereby the angiography dye is introduced in the blood vessel.

7. The method of claim 6 wherein the plurality of apertures are located adjacent and upstream of the occlusion balloon, short of the sheath distal end so that during the introducing step, angiography dye does not flow downstream with a blood flow in the blood vessel.

8. The method of claim 6 comprising:
providing an inflation lumen within the introducer sheath and conveying fluid through the inflation lumen to the occlusion balloon during said inflating step;
providing an injection lumen within the introducer sheath distal from the inflation lumen; and
conveying the angiography dye through the injection lumen during said introducing step.

9. The method of claim 8 comprising providing a sheath internal lumen within the introducer sheath distal from the inflation lumen and the injection lumen, and maintaining the dilator in the sheath internal lumen during said inserting step.

10. The method of claim 9 comprising conveying the angiography dye through a passageway defined between a dilator exterior surface and a sheath interior surface, during said introducing step.

11. A directional sheath comprising:
an introducer sheath including a sheath distal end defining a sheath distal end opening, and a sheath proximal end, a port being joined with the sheath proximal end, the introducer sheath defining a plurality of apertures between the sheath distal end and the sheath proximal end, the plurality of apertures distal from the sheath distal end opening and distal from the port, the introducer sheath including an occlusion balloon located adjacent the sheath distal end, the introducer sheath including an inflation lumen in fluid communication with the occlusion balloon and the sheath proximal end; and
a dilator including a dilator distal end and a dilator proximal end, the dilator being selectively disposed in the introducer sheath.

12. The directional sheath of claim 11 comprising a sheath interior between the sheath distal end and sheath proximal end, the sheath interior having a sheath interior dimension, wherein the plurality of apertures provide fluid communication between the sheath interior and a sheath exterior which is open to a surrounding environment.

13. The directional sheath of claim 11,
wherein the dilator includes a dilator intermediate portion located between the dilator distal end and the dilator proximal end,
wherein the dilator is positioned within the introducer sheath so that the dilator distal end occludes the sheath distal end and so that liquid cannot flow out the sheath distal end opening,
wherein the dilator distal end has an enlarged head having a first dimension,
wherein the dilator intermediate portion has a second dimension, less than the first dimension and less than the sheath interior dimension, so that liquid can flow through the introducer sheath, between the dilator intermediate portion and the sheath interior, and out the plurality of apertures.

14. The directional sheath of claim 11 wherein the introducer sheath defines an internal sheath lumen bounded by an exterior sheath wall, the exterior sheath wall including a sheath exterior surface and a sheath interior surface, the internal sheath lumen being in fluid communication with the port, the exterior sheath wall defining the plurality of apertures.

15. The directional sheath of claim 11,
wherein the introducer sheath defines an internal sheath lumen bounded by an exterior sheath wall,
wherein the exterior sheath wall defines an injection lumen separate from the internal sheath lumen and the inflation lumen,
wherein the injection lumen is in fluid communication with the plurality of apertures,
whereby the injection lumen is adapted to convey angiography dye through the injection lumen during an angiography procedure.

16. The directional sheath of claim 11 wherein the sheath includes a fluid passageway in fluid communication with the plurality of apertures, whereby an angiography dye can flow through the fluid passageway to the plurality of apertures and out of the introducer sheath, into a blood vessel.

17. The directional sheath of claim 16 comprising a rotation limiter located adjacent the proximal end, wherein the rotation limiter is configured to stop rotation of the dilator when the dilator fluid passageway is aligned with the plurality of apertures.

18. The directional sheath of claim 11 wherein the dilator includes a dilator longitudinal axis, wherein the dilator defines a helical passageway around the dilator longitudinal axis, wherein the helical passageway is configured to enable liquid to flow adjacent the dilator, through the plurality of apertures and out of the introducer sheath, into a blood vessel.

19. The directional sheath of claim 11 wherein the dilator includes a dilator longitudinal axis, wherein the introducer sheath defines an internal sheath lumen bounded by an exterior sheath wall, wherein the internal sheath lumen includes an internal sheath lumen axis, that is coaxial with the dilator longitudinal axis.

20. The directional sheath of claim 11 wherein the introducer sheath defines an internal sheath lumen bounded by an exterior sheath wall, wherein the internal sheath lumen includes an internal sheath lumen axis, wherein the exterior sheath wall defines an injection lumen separate from the internal sheath lumen, the injection lumen having an injection lumen longitudinal axis that is offset and distal from the internal sheath lumen axis wherein the exterior sheath wall defines an inflation lumen separate from the internal sheath lumen, the inflation lumen having an inflation lumen longitudinal axis that is offset and distal from the internal sheath lumen axis.
